(19) United States
(12) Patent Application Publication
(10) Pub. No.: US 2008/0152604 A1
(43) Pub. Date: Jun. 26, 2008

(54) SKIN-LIGHTENING COMPOSITIONS WITH AN IMPROVED ACTION

(76) Inventors: Thomas DOERING, Dormagen (DE); Armin WADLE, Erkrath (DE); Marianne WALDMANN-LAUE, Monheim (DE); Bernd ANDERHEGGEN, Haan (DE)

Correspondence Address: PAUL & PAUL, 2000 MARKET STREET, PHILADELPHIA, PA, 19103-3229, US

(21) Appl. No.: 11/968,401

(22) Filed: Jan. 2, 2008

Related U.S. Application Data
(63) Continuation of application No. PCT/EP2006/006109, filed on Jun. 24, 2006.

(30) Foreign Application Priority Data
Jul. 4, 2005 (DE) 10 2005 031 482.1

(51) Int. Cl. A61K 8/36; A61Q 19/02; A61Q 17/04

(52) U.S. Cl. 424/60; 424/62

(57) ABSTRACT

Cosmetic or dermatological preparations are comprised of from 0.01 to 5% by weight of a dicarboxylic acid selected from the group consisting of 8-hexadecene-1,16-dicarboxylic acid decanedioic acid nonanedioic acid and combinations thereof and from 0.01 to 5% by weight of ascorbic acid, salts of ascorbic acid, ascorbic acid derivatives and combinations thereof. These preparations provide skin-lightening and/or hair-lightening preparations which inhibit melanin biosynthesis and do not damage hair and/or skin.

### CROSS-REFERENCE TO RELATED APPLICATIONS

This application is a continuation under 35 U.S.C. Section 365(c) and 35 U.S.C. Section 120 of International Application No. PCT/EP2006/006109, filed Jun. 24, 2006. This application also claims priority under 35 U.S.C. Section 119 of German Patent Application No. DE 10 2005 031 482.1, filed Jul. 4, 2005. Both the International Application and the German Application are incorporated herein by reference in their entireties.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The present invention relates to cosmetic or dermatological compositions for the cosmetic and topical dermatological lightening of skin or for the prevention of skin tanning, more particularly of skin tanning induced by UV radiation, and also for lightening keratinic fibers, more particularly, the natural color of human hair.

The pigmentation of the skin is accomplished through melanocytes, which are encountered in the lowest layer of the epidermis, alongside the basal cells, as pigment-forming cells which, depending on skin type, occur either in isolation or in clusters. Melanocytes contain melanosomes, in which melanin is formed. A variety of chemical and/or physical influences, UV radiation more particularly, result in increased melanin formation. This melanin is transported via the keratinocytes into the corneocytes (horny layer), and leads to a brownish to brown-black skin color. Melanin is formed as the end stage of an oxidative process in which tyrosine, with the participation of the enzyme tyrosinase, is converted via a plurality of intermediates into the brown to brown-black eumelanins (DHICA melanin and DHI melanin) and/or, with the participation of sulfur compounds, into the reddish pheomelanin.

In the same way as for the pigmentation of the skin, melanin-producing melanocytes are likewise responsible for the color of hair. The quantity and composition of the melanin in the hair defines the natural hair color, which is genetically determined.

The formation of melanin—and hence the color of skin and hair—is subject to external influences as well as desired effects and (“healthy tan”) may also lead to unwanted phenomena. Thus, for example, UV radiation may lead to freckles. Instances of aberrant pigmentation may also occur on the basis of genetic disposition, wound healing or scarring, or skin aging (“age spots”).

Since the process of desquamation (superficial detachment of cells or groups of cells from their epithelial association) entails continual loss of melanin, skin lightening can be achieved through inhibition of the synthesis of new melanin.

(2) Description of Related Art, Including Information Disclosed Under 37 C.F.R. Sections 1.97 and 1.98.

Effective cosmetic preparations for skin lightening are known. In the majority of cases, however, the active they contain is hydroquinone or kojic acid (inhibition of tyrosinase). Both substances are mutagenic and therefore should not be used over prolonged periods.

The color of hair is lightened mostly by employing oxidative processes, the best-known of which is the bleaching of hair with hydrogen peroxide. These processes, however, may result in damage to the hair.

Therefore, the object was to provide skin-lightening and/or hair-lightening preparations which are free from the stated drawbacks.

### BRIEF SUMMARY OF THE INVENTION

It has emerged, surprisingly, that the combination of 8-hexadecene-1,16-dicarboxylic acid with vitamin C or derivatives of vitamin C (for example, ascorbyl phosphate, ascorbyl glucoside, ascorbyl tetraisopalmitate) leads to a synergistic inhibition of melanin synthesis. When applied extensively to the skin, particularly in the case of Indians and Asians, this leads to a lightening of the skin color. On local application to pigment defects such as age spots, these defects lose their contrast relative to the bordering area of the skin, and are therefore less visible.

### BRIEF DESCRIPTION OF THE SEVERAL VIEWS OF THE DRAWING(S)

Not Applicable

### DETAILED DESCRIPTION OF THE INVENTION

The present invention provides in a first embodiment a cosmetic or dermatological preparation which contains 0.01% to 5% by weight of 8-hexadecene-1,16-dicarboxylic acid and/or decanedioic acid (sebacic acid) and/or nonanedioic acid (azelaic acid) and 0.01% to 5% by weight of ascorbic acid and/or salts of ascorbic acid and/or ascorbic acid derivatives.

The compositions of the invention contain 0.01% to 5% by weight of 8-hexadecene-1,16-dicarboxylic acid and/or decanedioic acid (sebacic acid) and/or nonanedioic acid (azelaic acid).

Decanedioic acid (sebacic acid), HOOC—(CH₂)₈—COOH, is sold in the form of colorless, monoclinic-prismatic leaflets and is prepared by heating castor oil or ricinoleic acid with NaOH and air or by Kolbe synthesis from the monomethyl ester of adipic acid, or by oxidation of cyclodecanol. Nonanedioic acid (azelaic acid), HOOC—(CH₂)₇—COOH, is likewise sold in the form of colorless leaflets or needles, and is prepared by oxidation from ricinoleic acid, or by ozonolysis of oleic acid.

8-Hexadecene-1,16-dicarboxylic acid (dioic acid, provisional INCI name Octadecenedioic acid), which is also referred to as 9-octadecene-1,18-dioic acid (see, for example, ROMPP Chemielexikon on the nomenclature of dicarboxylic acids), is a metabolic product of yeast cells from selected mutant strains of the Candida strain, the starting substance being a fatty acid of purely plant origin, which is converted into the hydroxyl-fatty acid, which is subsequently oxidized via the stage of the fatty acid aldehyde to give the dicarboxy acid. The commercial product possesses a purity of 95%, in which the 8-hexadecene-1,16-dicarboxylic acid is in the form of a mixture of the cis and trans isomers, and the cis isomer predominates in terms of amount. The product can contain up to 3% by weight of oleic acid.

Preferred cosmetic or dermatological preparations of the invention are characterized in that they contain, based on their weight, 0.05% to 3.0% by weight, preferably, 0.07% to 2.0% by weight, and more particularly, 0.1% to 1.0% by weight of 8-hexadecene-1,16-dicarboxylic acid.

The preparations of the invention may constitute, for example, a solution, an emulsion of the water-in-oil (W/O) type or of the oil-in-water (O/W) type, or a multiple emulsion, of the water-in-oil-in-water (W/O/W) or oil-in-water-in-oil (O/W/O) type, for example, an aqueous dispersion or lipoid dispersion, a gel, a solid stick, a transdermal therapeutic system or else an aerosol.

Emulsions of the invention in the sense of the present invention, in the form for example, of a cream, a lotion or a cosmetic milk, are advantageous and comprise, for example, fats, oils, waxes and/or other lipids, and also water and one or more emulsifiers, of the kind typically used for a formulation of this type.

It is also possible and advantageous in the sense of the present invention to provide the preparations of the invention as aqueous systems or surfactant preparations for the cleansing and care of the skin and the hair. This encompasses not only shower gels and shampoos but also conditioners, hair treatments, hair rinses, hair tonics, sprays, etc.

As a further essential ingredient the compositions of the invention comprise ascorbic acid and/or ascorbic acid derivatives.

Ascorbic acid {(R)-5-[(S)-1,2-dihydroxyethyl]-3,4-dihydroxy-5H-furan-2-one, also called vitamin C} is the oldest-known vitamin. L-Ascorbic acid is an enediol and has a strongly reducing action. As a vinylog acid, L-ascorbic acid forms stable salts with metals. With its alcoholic hydroxyl groups, it forms esters with fatty acids, e.g., L-ascorbyl palmitate. Besides L-ascorbic acid, the most important commercial forms are the water-soluble salts sodium L-ascorbate, calcium L-ascorbate, and the fat-soluble L-ascorbyl palmitate. The enediol group at C2 and C3 readily undergoes transition to the 2,3-dioxo group. Dehydroascorbic acid, as the primary oxidation product, forms a redox system with ascorbic acid. The lactone ring of the dehydroascorbic acid can be hydrolyzed, forming dioxogulonic acid, which has no vitamin activity. Reformation of ascorbic acid is no longer possible. The majority of plant and animal species are capable of their own L-ascorbic acid biosynthesis, but humans, primates, and guinea pigs are not. In certain foods, such as sauerkraut or brassicas, L-ascorbic acid may be present attached to glucosinolates, in the form of the ascorbigens, which do not have vitamin activity:

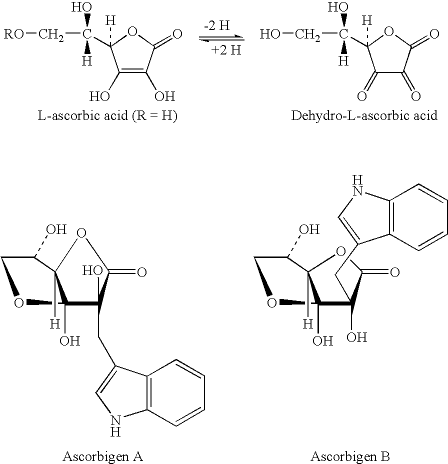

Industrially L-ascorbic acid is prepared in a combination of a microbial and two or more subsequent chemical reactions. The starting compound is D-sorbitol, which is dehydrogenated by fermentation with the bacterium Acetobacter suboxydans to give the keto sugar L-sorbose. Sorbose is then converted by chemical oxidation into 2-oxo-L-gulonic acid, from which the sodium salt of the enol compound comes about in alkaline solution. When this enol compound is acidified, it turns directly into L-ascorbic acid.

Particularly preferred cosmetic or dermatological preparations of the invention are characterized in that they contain, based on their weight, 0.05% to 3.0% by weight, preferably 0.07% to 2.0% by weight, and more particularly 0.1% to 1.0% by weight of ascorbic acid and/or salts of ascorbic acid and/or ascorbic acid derivatives.

Suitable ascorbic acid derivatives are more particularly the salts of ascorbic acid, i.e., hydroascorbates and ascorbates, among which the alkali metal salts and alkaline earth metal salts are preferred. Particularly preferred salts of ascorbic acid which can be used in accordance with the invention are sodium hydroascorbate, sodium ascorbate, potassium hydroascorbate, potassium ascorbate, ammonium ascorbate, ammonium hydroascorbate, calcium ascorbate, calcium hydroascorbate, magnesium ascorbate, and magnesium hydroascorbate.

With particular preference it is also possible to use the esters of ascorbic acid (R#H in the formula set out above). Among the esters of ascorbic acid preference is given more particularly to ascorbyl methanoate, ascorbyl ethanoate, ascorbyl n-propanoate, ascorbyl isopropanoate, ascorbyl n-butanoate, etc. With particular preference the compositions of the invention comprise esters of ascorbic acid with fatty acids, more particularly ascorbyl linoleate, ascorbyl oleate, ascorbyl oleinate, ascorbyl palmitate, ascorbyl stearate, etc. Ascorbyl oleate (6-palmitoyl-L-ascorbic acid)

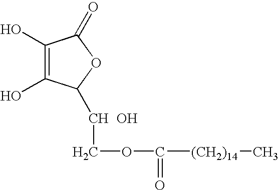

is particularly preferred in accordance with the invention, as is ascorbyl tetraisopalmitate.

“Inorganic” acids as well can be esterified with ascorbic acid. Preferred among the “inorganic” esters of ascorbic acid is, more particularly, ascorbyl phosphate.

Ascorbic acid derivatives with glycosidically attached sugars as well can be employed with particular preference in accordance with the invention. A derivative which has proven itself more particularly preferred here is ascorbyl glucoside.

In summary, preferred cosmetic or dermatological preparations of the invention are those which comprise ascorbyl phosphate and/or ascorbyl glucoside and/or ascorbyl tetraisopalmitate.

It is preferred to use 8-hexadecene-1,16-dicarboxylic acid and ascorbic acid and/or ascorbic acid derivative(s) in defined proportions to one another. Preference is given here to cosmetic or dermatological preparations of the invention wherein the weight ratio of 8-hexadecene-1,16-dicarboxylic acid to ascorbic acid and/or salts of ascorbic acid and/or ascorbic acid derivatives is 5:1 to 1:15, preferably 2:1 to 1:5, and more particularly 2:1 to 1:1.

The aforementioned weight ratios refer to the ratios formed from the amount of 8-hexadecene-1,16-dicarboxylic acid and the amount of ascorbic acid and all ascorbic acid derivatives that may be present in the composition.

The preparations of the invention may comprise further ingredients of cosmetics, which are described below. Preference in accordance with the invention, however, is also given to not incorporating certain ingredients into the preparations of the invention. Here, preference is given more particularly to cosmetic or dermatological preparations of the invention which contain no retinol and no retinol derivatives.

Retinol (vitamin A₁) belongs, together with 3,4-didehydroretinol (vitamin A₂), to the group of substances referred to as vitamin A. β-Carotene is the provitamin of retinol. The compositions of the invention are preferably free from these substances, in other words, containing neither retinol nor 3,4-didehydroretinol nor β-carotene nor vitamin A acid and its esters, vitamin A aldehyde and vitamin A alcohol, and also its esters such as the palmitate and the acetate.

Preferred ingredients which may be included in the preparations of the invention are, for example, surfactants and/or emulsifiers, fragrances, dyes, etc., which are described in detail below.

Cosmetic or dermatological preparations particularly preferred in accordance with the invention are characterized in that they contain, based on their weight, 0.5% to 20.0% by weight, preferably 1.0% to 15.0% by weight, and more particularly 2.0% to 10.0% by weight of propylene glycol isostearate.

Cosmetic or dermatological preparations of the invention that are further preferred contain, based on their weight, 0.5% to 20.0% by weight, preferably 1.0% to 15% by weight, of one or more compounds from the group consisting of triethylhexanone, glyceryl isostearate, propylene glycol isostearate, isopropyl isostearate, ethylhexyl palmitate, ethylhexyl isostearate, squalane, and triisostearin.

Likewise preferred is the use of taurine. Preference is given here to cosmetic or dermatological preparations of the invention which further contain 0.5% to 2.5% by weight, preferably 0.75% to 1.5% by weight, and more particularly 1% to 1.25% by weight of 2-aminoethanesulfonic acid (taurine).

It is also advantageous in the sense of the present invention to formulate cosmetic or dermatological preparations whose primary purpose is not to protect against sunlight, but which nevertheless include further UV protectants.

The UV filters to be used according to the invention are not subject to any general limitations with regard to their structure and their physical properties. Rather, all UV filters which can be used in the cosmetics sector and whose absorption maximum is in the UVA(315-400 nm) region, in the UVB(280-315 nm) region or in the UVC(<280 nm) region are suitable. UV filters with an absorption maximum in the UVB region, in particular, in the range from about 280 to about 300 nm, are particularly preferred.

The UV filters used according to the invention can be selected, for example, from substituted benzophenones, p-aminobenzoic acid esters, diphenylacrylic acid esters, cinnamic acid esters, salicylic acid esters, benzimidazoles and o-aminobenzoic acid esters.

Examples of UV filters which can be used according to the invention are 4-aminobenzoic acid, N,N,N-trimethyl-4-(2-oxoborn-3-ylidenemethyl)aniline methylsulfate, 3,3,5-trimethylcyclohexyl salicylate (homosalate), 2-hydroxy-4-methoxybenzophenone (benzo-phenone-3; Uvinul®M 40, Uvasorb®MET, Neo Heliopan®BB, Eusolex®4360), 2-phenyl-benzimidazole-5-sulfonic acid and the potassium, sodium and triethanolamine salts thereof (phenylbenzimidazolesulfonic acid; Parsol®HS; Neo Heliopan®Hydro), 3,3′-(1,4-phenylenedimethylene)bis(7,7-dimethyl-2-oxobicyclo[2.2.1]hept-1-ylmethanesulfonic acid) and salts thereof, 1-(4-tert-butylphenyl)-3-(4-methoxyphenyl)propane-1,3-dione (butyl methoxydibenzoylmethane; Parsol®1789, Eusolex®9020), α-(2-oxoborn-3-ylidene)toluene-4-sulfonic acid and salts thereof, ethoxylated ethyl 4-aminobenzoate (PEG-25 PABA; Uvinul®P 25), 2-ethylhexyl 4-dimethylaminobenzoate (octyl dimethyl PABA; Uvasorb®DMO, Escalol®507, Eusolex®6007), 2-ethylhexyl salicylate (octyl salicylate; Escalol®587, Neo Heliopan®OS, Uvinul®018), isopentyl 4-methoxycinnamate (isoamyl p-methoxycinnamate; Neo Heliopan®E 1000), 2-ethylhexyl 4-methoxycinnamate (octyl methoxycinnamate; Parsol®MCX, Escalol®557, Neo Heliopan®AV), 2-hydroxy-4-methoxybenzophenone-5-sulfonic acid and sodium salt thereof (benzophenone-4; Uvinul®MS 40; Uvasorb®S 5), 3-(4′-methylbenzylidene)-D,L-camphor (4-methyl-benzylidenecamphor; Parsol®5000, Eusolex®6300), 3-benzylidenecamphor, 4-isopropylbenzyl salicylate, 2,4,6-trianilino(p-carbo-2′-ethylhexyl-1′-oxy)-1,3,5-triazine, 3-imidazol-4-ylacrylic acid and the ethyl ester thereof, polymers of N-{(2 and 4)-[2-oxoborn-3-ylidenemethyl)benzyl}acrylamide, 2,4-dihydroxybenzophenone (benzophenone-1; Uvasorb®20 H, Uvinul®400), 1,1′-diphenylacrylonitrile acid 2-ethylhexyl ester (octocrylene; Eusolex®OCR, Neo Heliopan®Type 303, Univul® N 539 SG), menthyl o-aminobenzoate (menthyl anthranilate; Neo Heliopan®MA), 2,2′,4,4′-tetrahydroxybenzophenone (benzophenone-2; Uvinul®D-50), 2,2′-dihydroxy-4,4′-dimethoxybenzophenone (benzophenone-6), 2,2′-dihydroxy-4,4′-dimethoxybenzophenone-5 sodium sulfonate and 2′-ethylhexyl 2-cyano-3,3-diphenylacrylate. Preference is given to 4-aminobenzoic acid, N,N,N-trimethyl-4-(2-oxoborn-3-ylidenemethyl)aniline methylsulfate, 3,3,5-trimethylcyclohexyl salicylate, 2-hydroxy-4-methoxy-benzophenone, 2-phenylbenzimidazole-5-sulfonic acid and the potassium, sodium and triethanolamine salts thereof, 3,3′-(1,4-phenylenedimethylene)bis(7,7-dimethyl-2-oxobicyclo[2.2.1]hept-1-ylmethanesulfonic acid) and salts thereof, 1-(4-tert-butylphenyl)-3-(4-methoxyphenyl)propane-1,3-dione, α-(2-oxoborn-3-ylidene)toluene-4-sulfonic acid and salts thereof, ethoxylated ethyl 4-aminobenzoate, 2-ethyl hexyl 4-dimethylaminobenzoate, 2-ethylhexyl salicylate, isopentyl 4-methoxycinnamate, 2-ethylhexyl 4-methoxycinnamate, 2-hydroxy-4-methoxybenzophenone-5-sulfonic acid and the sodium salt thereof, 3-(4′-methylbenzylidene)-D,L-camphor, 3-benzylidenecamphor, 4-isopropylbenzylsalicylate, 2,4,6-trianilino(p-carbo-2′-ethylhexyl-1′-oxy)-1,3,5-triazine, 3-imidazol-4-ylacrylic acid and the ethyl ester thereof, polymers of N-{(2 and 4)-[2-oxoborn-3-ylidenemethyl]benzyl}acrylamide. According to the invention, very particular preference is given to 2-hydroxy-4-methoxybenzophenone, 2-phenylbenzimidazole-5-sulfonic acid and the potassium, sodium and triethanolamine salts thereof, 1-(4-tert-butylphenyl)-3-(4-methoxyphenyl)propane-1,3-dione, 2-ethylhexyl 4-methoxycinnamate and 3-(4′-methylbenzylidene)-D,L-camphor.

Preference is given to those UV filters whose molar extinction coefficient at the absorption maximum is above 15,000, in particular, above 20,000.

Furthermore, it has been found that for structurally similar UV filters, in many cases the water-insoluble compound within the scope of the teaching according to the invention has the higher effect compared to those water-soluble compounds which differ from it by virtue of one or more additional ionic groups. For the purposes of the invention, water-insoluble UV filters are to be understood as meaning those which dissolve in water at 20° C. to not more than 1% by weight, in particular, to not more than 0.1% by weight. Furthermore, these compounds should be soluble in customary cosmetic oil components at room temperature to at least 0.1% by weight, in particular, to at least 1% by weight. The use of water-insoluble UV filters can therefore be preferred according to the invention.

According to a further embodiment of the invention, preference is given to those UV filters which have a cationic group, in particular, a quaternary ammonium group.

These UV filters have the general structure U-Q.

The structural moiety U is here a group which absorbs UV rays. This group can, in principle, be derived from the known above-mentioned UV filters which can be used in the cosmetics sector by replacing one group, generally a hydrogen atom, of the UV filter with a cationic group Q, in particular, with a quaternary amino function.

Compounds from which the structural moiety U can be derived are, for example,
substituted benzophenones,
p-aminobenzoic acid esters,
diphenylacrylic acid esters,
cinnamic acid esters,
salicylic acid esters,
benzimidazoles and
o-aminobenzoic acid esters.

Structural moieties U which are derived from cinnamamide or from N,N-dimethylaminobenzoamide are preferred according to the invention.

The structural moieties U can in principle be chosen so that the absorption maximum of the UV filters can be both in the UVA(315-400 nm) region, and in the UVB(280-315 nm) region or in the UVC(<280 nm) region. UV filters with an absorption maximum in the UVB region, in particular, in the region from about 280 to about 300 nm, are particularly preferred.

In addition, the structural moiety U is chosen, also depending on structural moiety Q, preferably such that the molar extinction coefficient of the UV filter at the absorption maximum is above 15,000, in particular, above 20,000.

The structural moiety Q comprises, as cationic group, preferably a quaternary ammonium group. This quaternary ammonium group can in principle be joined directly to the structural moiety U, meaning that the structural moiety U is one of the four substituents of the positively charged nitrogen atom. However, one of the four substituents on the positively charged nitrogen atom is preferably a group, in particular, an alkylene group having 2 to 6 carbon atoms, which functions as linkage between the structural moiety U and the positively charged nitrogen atom.

Advantageously, the group Q has the general structure —(CH₂)ₓ—N⁺R¹R²R³X⁻, in which x is an integer from 1 to 4, R¹ and R², independently of one another, are C₁₋₄ alkyl groups, R³ is a C₁₋₂₂ alkyl group or a benzyl group and X⁻ is a physiologically compatible anion. Within the context of this general structure, x is preferably 3, R¹ and R² are in each case a methyl group and R³ is either a methyl group or a saturated or unsaturated, linear or branched hydrocarbon chain having 8 to 22, in particular, 10 to 18, carbon atoms.

Physiologically compatible anions are, for example, inorganic anions, such as halides, in particular, chloride, bromide and fluoride, sulfate ions and phosphate ions, and organic anions, such as lactate, citrate, acetate, tartrate, methosulfate and tosylate.

Two preferred UV filters with cationic groups are the commercially available compounds cinnamamidopropyltrimethylammonium chloride (Incroquat®UV-283) and dodecyldimethylaminobenzamidopropyldimethylammonium tosylate (Escalol®HP 610).

Of course the teaching according to the invention also includes the use of a combination of two or more UV filters. Within the scope of this embodiment, the combination of at least one water-insoluble UV filter with at least one UV filter with a cationic group is preferred.

The UV filters (I) are present in the compositions according to the invention preferably in amounts of 0.1-5% by weight, based on the overall composition. Amounts of 0.4-2.5% by weight are particularly preferred.

A further preferred substance which can be incorporated into the preparations of the invention is ethylhexyltriazone. Particular preference is given here to cosmetic or dermatological preparations of the invention which further contain 0.5% to 2.5% by weight, preferably 0.75% to 1.5% by weight, of ethylhexyltriazone.

Further preferred cosmetic or dermatological preparations of the invention are characterized in that they further contain 0.5% to 2.5% by weight, preferably, 0.75% to 1.5% by weight, of (UVA)butylmethoxydibenzoylmethane (BMDBM) and/or diethylaminohydroxybenzoylhexyl benzoate.

Inorganic pigments as well can be employed in accordance with the invention as UV filters. Preferred inorganic pigments are metal oxides and/or other metal compounds of low or zero solubility in water, examples being carbonates, sulfates, etc. Preference is given more particularly to oxides of titanium (TiO₂), of zinc (ZnO), of iron (e.g., Fe₂O₃), of zirconium (ZrO₂), of silicon (SiO₂), of manganese (e.g., MnO), of aluminum (Al₂O₃), of cerium (e.g., Ce₂O₃), mixed oxides of the corresponding metals, and also blends of such oxides, and also the sulfate of barium (BaSO₄).

The pigments can also be employed in the form of commercially available oily or aqueous preliminary dispersions. These preliminary dispersions may advantageously have had dispersing assistants and/or solubilization mediators added to them.

The pigments may, where appropriate, have been superficially treated (“coated”), the intention being, for example, that a hydrophilic, amphiphilic or hydrophobic character should be formed or retained. This surface treatment may involve giving the pigments a thin hydrophilic and/or hydrophobic organic and/or inorganic coat by methods which are known per se. The various surface coatings may also comprise water.

Inorganic surface coatings may be composed, for example, of aluminum oxide (Al₂O₃), aluminum hydroxide AI(OH)₃, or aluminum oxide hydrate, sodium hexametaphosphate (NaPO₃)₆, sodium metaphosphate (NaPO₃)ₙ, silicon dioxide (SiO₂) or iron oxide (Fe₂O₃). These inorganic surface coatings may occur alone, in combination and/or in combination with organic coating materials.

Organic surface coatings may comprise vegetable or animal aluminum stearate, vegetable or animal stearic acid, lauric acid, dimethylpolysiloxane, methylpolysiloxane, simethicone (a mixture of dimethylpolysiloxane having an average chain length of 200 to 350 dimethylsiloxane units and silica gel) or alginic acid. These organic surface coatings may occur alone, in combination and/or in combination with inorganic coating materials.

Preferably in accordance with the invention it is possible to employ titanium dioxide. Preference is given here to cosmetic or dermatological preparations of the invention which further contain 0.1% to 2.5% by weight, preferably, 0.5% to 1.0% by weight, of titanium dioxide.

The compositions of the invention may include further actives and auxiliaries. These ingredients are described below.

The use of surfactants (E) in the compositions according to the invention has proven particularly advantageous. Therefore, in a further preferred embodiment, the compositions according to the invention comprise surfactants. The term “surfactants” is understood as meaning interface-active substances which can form adsorption layers at surfaces and interfaces or can aggregate in volume phases to give micelle colloids or lyotropic mesophases. A distinction is made between anionic surfactants consisting of a hydrophobic radical and a negatively charged hydrophilic head group, amphoteric surfactants, which carry both a negative and a compensating positive charge, cationic surfactants, which have a positively charged hydrophilic group besides a hydrophobic radical, and nonionic surfactants, which have no charges but strong dipole moments and are highly hydrated in aqueous solution.

Suitable anionic surfactants (E1) in preparations according to the invention are all anionic surface-active substances suitable for use on the human body. These are characterized by a water-solubilizing, anionic group such as, for example, a carboxylate group, sulfate group, sulfonate group or phosphate group, and a lipophilic alkyl group having about 8 to 30 carbon atoms. In addition, glycol or polyglycol ether groups, ester groups, ether groups and amide groups, and hydroxyl groups may be present in the molecule. Examples of suitable anionic surfactants are in each case in the form of the sodium, potassium and ammonium salts, and the mono-, di- and trialkanolammonium salts having 2 to 4 carbon atoms in the alkanol group,
linear and branched fatty acids having 8 to 30 carbon atoms (soaps),
ether carboxylic acids of the formula
R—O—(CH₂—CH₂O)ₓ—CH₂—COOH, in which R is a linear alkyl group having 8 to 30 carbon atoms and x is 0 or 1 to 16,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic mono- and dialkyl esters having 8 to 24 carbon atoms in the alkyl group and sulfosuccinic monoalkylpolyoxyethyl esters having 8 to 24 carbon atoms in the alkyl group and 1 to 6 oxyethyl groups,
linear alkanesulfonates having 8 to 24 carbon atoms,
linear alpha-olefinsulfonates having 8 to 24 carbon atoms,
alpha-sulfo fatty acid methyl esters of fatty acids having 8 to 30 carbon atoms,
alkyl sulfates and alkyl polyglycol ether sulfates of the formula R—O(CH₂—CH₂O)ₓ—OSO₃H, in which R is a preferably linear alkyl group having 8 to 30 carbon atoms and x is 0 or 1 to 12,
sulfated hydroxyalkyl polyethylene and/or hydroxyalkylene propylene glycol ethers
sulfonates of unsaturated fatty acids having 8 to 24 carbon atoms and 1 to 6 double bonds,
esters of tartaric acid and citric acid with alcohols, which are addition products of about 2-15 molecules of ethylene oxide and/or propylene oxide onto fatty alcohols having 8 to 22 carbon atoms,
alkyl and/or alkenyl ether phosphates of the formula (E1-I)

R¹(OCH₂CH₂)ₙ—O—P(O)(OX)—OR² (E1-I)
in which R¹ is preferably an aliphatic hydrocarbon radical having 8 to 30 carbon atoms, R² is hydrogen, a radical (CH₂CH₂O)ₙR² or X, n is numbers from 1 to 10 and X is hydrogen, an alkali metal or alkaline earth metal or NR³R⁴R⁵R⁵, where R³ to R⁶, independently of one another, are hydrogen or a C₁ to C₄ hydrocarbon radical,
sulfated fatty acid alkylene glycol esters of the formula (E1-II)

R⁷CO(AlkO)ₙSO₃M (E1-II)
in which R⁷CO— is a linear or branched, aliphatic, saturated and/or unsaturated acyl radical having 6 to 22 carbon atoms, Alk is CH₂CH₂, CHCH₃CH₂ and/or CH₂CHCH₃, n is numbers from 0.5 to 5 and M is a cation,
monoglyceride sulfates and monoglyceride ether sulfates of the formula (E1-III)

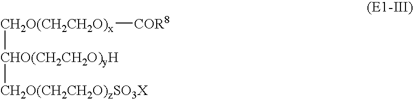

in which R⁸CO is a linear or branched acyl radical having 6 to 22 carbon atoms, x, y and z are in total 0 or numbers from 1 to 30, preferably 2 to 10, and X is an alkali metal or alkaline earth metal. Typical examples of monoglyceride (ether) sulfates suitable for the purposes of the invention are the reaction products of lauric acid monoglyceride, coconut fatty acid monoglyceride, palmitic acid monoglyceride, stearic acid monoglyceride, oleic acid monoglyceride and tallow fatty acid monoglyceride, and ethylene oxide adducts thereof with sulfur trioxide or chlorosulfonic acid in the form of their sodium salts. Preference is given to using monoglyceride sulfates of the formula (E1-III) in which R³CO is a linear acyl radical having 8 to 18 carbon atoms,
amide ether carboxylic acids,
condensation products of C₈-C₃₀ fatty alcohols with protein hydrolyzates and/or amino acids and derivatives thereof, which are known to the person skilled in the art as protein fatty acid condensates, such as, for example, the Lamepon® grades, Gluadin® grades, Hostapon® KCG or the Amisoft® grades.

Preferred anionic surfactants are alkyl sulfates, alkyl polyglycol ether sulfates and ether carboxylic acids having 10 to 18 carbon atoms in the alkyl group and up to 12 glycol ether groups in the molecule, sulfosuccinic acid mono- and dialkyl esters having 8 to 18 carbon atoms in the alkyl group and sulfosuccinic acid monoalkylpolyoxyethyl esters having 8 to 18 carbon atoms in the alkyl group and 1 to 6 oxyethyl groups, monoglyceride sulfates, alkyl and alkenyl ether phosphates, and protein fatty acid condensates.

Zwitterionic surfactants (E2) is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example, cocoalkyldimethylammonium glycinate, N-acylaminopropyl-N,N-dimethylammonium glycinates, for example, cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethyl hydroxyethylcarboxymethyl glycinate. A preferred zwitterionic surfactant is the fatty acid amide derivative known under the INCI name Cocamidopropyl Betaine.

Ampholytic surfactants (E3) are understood as meaning those surface-active compounds which, apart from a C₈-C₂₄-alkyl or -acyl group in the molecule, comprise at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamido-propylglycines, N-alkyltaurines, N-alkylsarcosines, 2-alkylaminopropionic acids and alkylaminoacetic acids having in each case about 8 to 24 carbon atoms in the alkyl group. Particularly preferred ampholytic surfactants are N-cocoalkylaminopropionate, cocoacylaminoethylaminopropionate and C₁₂-C₁₈-acylsarcosine.

Nonionic surfactants (E4) comprise as hydrophilic group, e.g., a polyol group, a polyalkylene glycol ether group or a combination of polyol and polyglycol ether group. Such compounds are, for example,
addition products of from 2 to 50 mol of ethylene oxide and/or 0 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group,
addition products, terminally capped with a methyl or C₂-C₆-alkyl radical, of from 2 to 50 mol of ethylene oxide and/or 0 to 5 mol of propylene oxide onto linear and branched fatty alcohols having 8 to 30 carbon atoms, onto fatty acids having 8 to 30 carbon atoms and onto alkylphenols having 8 to 15 carbon atoms in the alkyl group, such as, for example, the grades available under the brand names Dehydrol® LS, Dehydrol® LT (Cognis),
C₁₂-C₃₀ fatty acid mono- and diesters of addition products of from 1 to 30 mol of ethylene oxide onto glycerol,
addition products of from 5 to 60 mol of ethylene oxide onto castor oil and hydrogenated castor oil,
polyol fatty acid esters, such as, for example, the commercial product Hydagen® HSP (Cognis) or Sovermol grades (Cognis),
alkoxylated triglycerides,
alkoxylated fatty acid alkyl esters of the formula (E4-I)

R¹CO—(OCH₂CHR²)_{w}OR³ (E4-I)
in which R¹CO is a linear or branched, saturated and/or unsaturated acyl radical having 6 to 22 carbon atoms, R² is hydrogen or methyl, R³ is linear or branched alkyl radicals having 1 to 4 carbon atoms and w is numbers from 1 to 20,
amine oxides,
hydroxy mixed ethers,
sorbitan fatty acid esters and addition products of ethylene oxide onto sorbitan fatty acid esters, such as, for example, the polysorbates,
sugar fatty acid esters and addition products of ethylene oxide onto sugar fatty acid esters,
addition products of ethylene oxide onto fatty acid alkanolamides and fatty amines,
sugar surfactants of the type of the alkyl and alkenyl oligoglycosides according to formula (E4-II),

R⁴O—[G]ₚ (E4-II)
in which R⁴ is an alkyl or alkenyl radical having 4 to 22 carbon atoms, G is a sugar radical having 5 or 6 carbon atoms and p is numbers from 1 to 10. They can be obtained by the relevant methods of preparative organic chemistry.
The alkyl and alkenyl oligoglycosides can be derived from aldoses or ketoses having 5 or 6 carbon atoms, preferably from glucose. The preferred alkyl and/or alkenyl oligoglycosides are thus alkyl and/or alkenyl oligoglucosides. The index number p in the general formula (E4-II) indicates the degree of oligomerization (DP), i.e. the distribution of mono- and oligoglycosides, and is a number between 1 and 10. While p in the individual molecule must always be an integer and here can in particular, assume values p=1 to 6, the value p for a specific alkyl oligoglycoside is an analytically determined arithmetic variable which in most cases is a fraction. Preference is given to using alkyl and/or alkenyl oligoglycosides with an average degree of oligomerization p of from 1.1 to 3.0. From an applications point of view, preference is given to those alkyl and/or alkenyl oligoglycosides whose degree of oligomerization is less than 1.7 and in particular, is between 1.2 and 1.4. The alkyl or alkenyl radical R⁴ can be derived from primary alcohols having 4 to 11, preferably 8 to 10, carbon atoms. Typical examples are butanol, caproyl alcohol, caprylyl alcohol, capryl alcohol and undecyl alcohol, and technical-grade mixtures thereof, as are obtained, for example, during the hydrogenation of technical-grade fatty acid methyl esters or in the course of the hydrogenation of aldehydes from the Roelen's oxo synthesis. Preference is given to alkyl oligoglucosides of chain length C₈-C₁₀ (DP=1 to 3), which are produced as forerunning in the distillative separation of technical-grade C₈-C₁₈ coconut fatty alcohol and may be contaminated with a fraction of less than 6% by weight of the C₁₋₂ alcohol, and alkyl oligoglucosides based on technical-grade C_{9/11} oxo alcohols (DP=1 to 3). In addition, the alkyl or alkenyl radical R¹⁵ can also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical-grade mixtures thereof, which can be obtained as described above. Preference is given to alkyl oligoglucosides based on hydrogenated C_{12/14} coconut alcohol with a DP of from 1 to 3.
sugar surfactants of the type of fatty acid-N-alkylpolyhydroxyalkylamides, a nonionic surfactant of the formula (E4-III),

R⁵CO—NR⁶—[Z] (E4-III)

in which R⁵CO is an aliphatic acyl radical having 6 to 22 carbon atoms, R⁶ is hydrogen, an alkyl or hydroxyalkyl radical having 1 to 4 carbon atoms and [Z] is a linear or branched polyhydroxyalkyl radical having 3 to 12 carbon atoms and 3 to 10 hydroxyl groups. The fatty acid-N-alkylpolyhydroxyalkylamides are known substances which are usually obtained by reductive amination of a reducing sugar with ammonia, an alkylamine or an alkanolamine and subsequent acylation with a fatty acid, a fatty acid alkyl ester or a fatty acid chloride. Preferably, the fatty acid-N-alkylpolyhydroxyalkylamides are derived from reducing sugars having 5 or 6 carbon atoms, in particular, from glucose. The preferred fatty acid-N-alkylpolyhydroxyalkylamides are therefore fatty acid-N-alkylglucamides, as given by the formula (E4-IV):

R⁷CO—NR⁸—CH₂—(CHOH)₄—CH₂OH (E4-IV)
As fatty acid-N-alkylpolyhydroxyalkylamides, preference is given to using glucamides of the formula (E4-IV) in which R⁸ is hydrogen or an alkyl group and R⁷CO is an acyl radical of caproic acid, caprylic acid, capric acid, lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid or erucic acid or technical-grade mixtures thereof. Particular preference is given to fatty acid-N-alkylglucamides of the formula (E4-IV) which are obtained by reductive amination of glucose with methylamine and subsequent acylation with lauric acid or C12/14 coconut fatty acid or a corresponding derivative. In addition, the polyhydroxyalkylamides can also be derived from maltose and palatinose.

Preferred nonionic surfactants have proven to be the alkylene oxide addition products onto saturated linear fatty alcohols and fatty acids having in each case 2 to 30 mol of ethylene oxide per mole of fatty alcohol or fatty acid. Preparations with excellent properties are likewise obtained if they comprise fatty acid esters of ethoxylated glycerol as nonionic surfactants.

These compounds are characterized by the following parameters. The alkyl radical R comprises 6 to 22 carbon atoms and may either be linear or branched. Preference is given to primary linear and 2-position methyl-branched aliphatic radicals. Such alkyl radicals are, for example, 1-octyl, 1-decyl, 1-lauryl, 1-myristyl, 1-cetyl and 1-stearyl. Particular preference is given to 1-octyl, 1-decyl, 1-lauryl, 1-myristyl. When using so-called “oxo alcohols” as starting materials, compounds with an uneven number of carbon atoms in the alkyl chain predominate.

In addition, very particularly preferred nonionic surfactants are the sugar surfactants. These can be present in the compositions used according to the invention preferably in amounts of 0.1-20% by weight, based on the total composition. Amounts of 0.5-15% by weight are preferred, and very particular preference is given to amounts of 0.5-7.5% by weight.

The compounds with alkyl groups used as surfactant may each be uniform substances. However, it is generally preferred, when producing these substances, to start from native vegetable or animal raw materials, thus giving mixtures of substances with different alkyl chain lengths that are dependent on the respective raw material.

In the case of the surfactants which constitute addition products of ethylene oxide and/or propylene oxide onto fatty alcohols or derivatives of these addition products, it is possible to use either products with a “normal” homolog distribution or those with a narrowed homolog distribution. “Normal” homolog distribution is understood here as meaning mixtures of homologs which are obtained in the reaction of fatty alcohol and alkylene oxide using alkali metals, alkali metal hydroxides or alkali metal alkoxides as catalysts. Narrowed homolog distributions, on the other hand, are obtained if, for example, hydrotalcites, alkaline earth metal salts of ethercarboxylic acids, alkaline earth metal oxides, hydroxides or alkoxides are used as catalysts. The use of products with a narrowed homolog distribution may be preferred.

According to the invention, cationic surfactants of the quaternary ammonium compound type, the ester quat type and the amidoamine type can be used. Preferred quaternary ammonium compounds are ammonium halides, in particular, chlorides and bromides, such as alkyltrimethylammonium chlorides, dialkyldimethylammonium chlorides and trialkyl-methylammonium chlorides, e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, and the imidazolium compounds known under the INCI names Quaternium-27 and Quaternium-83. The long alkyl chains of the above-mentioned surfactants preferably have 10 to 18 carbon atoms.

According to the invention, preference is given to using QAV with behenyl radicals, in particular, the substances known under the name behentrimonium chloride or bromide (docosanyltrimethylammonium chloride or bromide). Other preferred QAVs have at least two behenyl radicals, where QAV which two behenyl radicals on an imidazolinium backbone are particularly preferred. These substances are commercially available, for example, under the names Genamin® KDMP (Clariant) and Crodazosoft® DBQ (Crodauza).

Ester quats are known substances which contain both at least one ester function and also at least one quaternary ammonium group as structural element. Preferred ester quats are quaternized ester salts of fatty acids with triethanolamine, quaternized ester salts of fatty acids with diethanolalkylamines and quaternized ester salts of fatty acids with 1,2-dihydroxypropyl-dialkylamines. Such products are sold, for example, under the trade names Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such ester quats.

The alkylamidoamines are usually prepared by amidation of natural or synthetic fatty acids and fatty acid cuts with dialkylaminoamines. One compound from this group of substances which is particularly suitable according to the invention is the stearamidopropyldimethylamine commercially available under the name Tegoamid® S 18.

The cationic surfactants are present in the compositions according to the invention preferably in amounts of 0.05% to 10% by weight, based on the overall composition. Amounts of 0.1% to 5% by weight are particularly preferred.

The surfactants (E) are used preferably in amounts of 0.1%-45% by weight, more preferably 0.5%-30% by weight and very particularly preferably of 0.5%-25% by weight, based on the overall composition according to the invention.

Anionic, nonionic, zwitterionic and/or amphoteric surfactants, and mixtures thereof, may be preferred according to the invention.

In summary, preference is given to cosmetic or dermatological preparations according to the invention which contain—based on their weight—0.5% to 70% by weight, preferably, 1% to 60% by weight, and more particularly, 5% to 25% by weight of anionic and/or nonionic and/or cationic and/or amphoteric surfactant(s).

A further preferred group of ingredients of the cosmetic or dermatological preparations according to the invention are vitamins, provitamins or vitamin precursors, which, as described above, preferably do not include retinol. They are described below.

The vitamin B group or the vitamin B complex includes, inter alia,
vitamin B₁ (thiamine),
vitamin B₂ (riboflavin),
vitamin B₃. This name often covers the compounds nicotinic acid and nicotinamide
(niacinamide). According to the invention, preference is given to nicotinamide, which is present in the compositions used according to the invention preferably, in amounts of from 0.05 to 1% by weight, based on the total composition.
vitamin B₅ (pantothenic acid, panthenol and pantolactone). Within the scope of this group, preference is given to using panthenol and/or pantolactone. Derivatives of the panthenol which can be used according to the invention are, in particular, the esters and ethers of panthenol, and cationically derivatized panthenols. Individual representatives are, for example, panthenol triacetate, panthenol monoethyl ether and its monoacetate, and the cationic panthenol derivatives disclosed in WO 92/13829. The specified compounds of the vitamin B₅ type are present in the compositions according to the invention preferably in amounts of 0.05%-10% by weight, based on the overall composition. Amounts of 0.1%-5% by weight are particularly preferred.
Vitamin B₆ (pyridoxine and pyridoxamine and pyridoxal).

Vitamin E (tocopherols, in particular, α-tocopherol). Tocopherol and its derivatives, which include, in particular, the esters, such as the acetate, the nicotinate, the phosphate and the succinate, are present in the compositions according to the invention, preferably, in amounts of 0.05-1% by weight, based on the overall composition.

Vitamin F. The term “vitamin F” is usually understood as meaning essential fatty acids, in particular, linoleic acid, linolenic acid and arachidonic acid.

Vitamin H. “Vitamin H” is the term used to refer to the compound (3aS,4S,6aR)-2-oxohexahydrothienol[3,4-d]imidazole-4-valeric acid, for which, however, the informal name biotin has meanwhile caught on. Biotin is present in the compositions according to the invention preferably in amounts of 0.001-0.5% by weight.

In summary, preference is given to cosmetic or dermatological preparations according to the invention which comprise vitamins, provitamins and vitamin precursors which are assigned to the groups B, C, E, F and H, where preferred compositions comprise the specified compounds in amounts of 0.1% to 5% by weight, preferably, of 0.25% to 4% by weight, and more particularly, 0.5% to 2.5% by weight, in each case based on the overall composition.

In a further preferred embodiment, the compositions according to the invention can comprise emulsifiers (F). At the phase interface, emulsifiers bring about the formation of water- or oil-stable adsorption layers which protect the dispersed droplets against coalescence and thus stabilize the emulsion. Emulsifiers, like surfactants, are therefore constructed from a hydrophobic molecular moiety and a hydrophilic molecular moiety. Hydrophilic emulsifiers form preferably O/W emulsions and hydrophobic emulsifiers form preferably W/O emulsions. An emulsion is to be understood as meaning a droplet-like distribution (dispersion) of one liquid in another liquid with the expenditure of energy to produce stabilizing phase interfaces by means of surfactants. The choice of these emulsifying surfactants or emulsifiers is governed here by the substances to be dispersed and the particular outer phase, and also the finely divided nature of the emulsion. Emulsifiers which can be used according to the invention are, for example,
addition products of from 4 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂-C₂₂ fatty acid mono- and diesters of addition products of from 1 to 30 mol of ethylene oxide onto polyols having 3 to 6 carbon atoms, in particular, onto glycerol,
ethylene oxide and polyglycerol addition products onto methyl glucoside fatty acid esters, fatty acid alkanolamides and fatty acid glucamides,
C₈-C₂₂-alkyl mono- and oligoglycosides and ethoxylated analogs thereof, where degrees of oligomerization of from 1.1 to 5, in particular, 1.2 to 2.0, and glucose as sugar component are preferred,
mixtures of alkyl(oligo)glucosides and fatty alcohols, for example, the commercially available product Montanov®68,
addition products of from 5 to 60 mol of ethylene oxide onto castor oil and hydrogenated castor oil,
partial esters of polyols having 3-6 carbon atoms with saturated fatty acids having 8 to 22 carbon atoms,
sterols. Sterols are understood as meaning a group of steroids which carry a hydroxyl group on carbon atom 3 of the steroid backbone and are isolated either from animal tissue (zoosterols) or from vegetable fats (phytosterols). Examples of zoosterols are cholesterol and lanosterol. Examples of suitable phytosterols are ergosterol, stigmasterol and sitosterol. Sterols are also isolated from fungi and yeasts, the so-called mycosterols.
phospholipids. This is understood, in particular, as meaning the glucose phospholipids, which are obtained, for example, as lecithins or phosphatidylcholines from, e.g., egg yolk or plant seeds (e.g., soya beans).
fatty acid esters of sugars and sugar alcohols, such as sorbitol,
polyglycerols and polyglycerol derivatives, such as, for example, polyglycerol poly-12-hydroxystearate (commercial product Dehymuls® PGPH),
linear and branched fatty acids having 8 to 30 carbon atoms and the Na, K, ammonium, Ca, Mg and Zn salts thereof.

The compositions according to the invention comprise the emulsifiers, preferably, in amounts of 0.1%-25% by weight, in particular, 0.5%-15% by weight, based on the overall composition.

Preferably, the compositions according to the invention can comprise at least one nonionogenic emulsifier with an HLB value of from 8 to 18. Nonionogenic emulsifiers with an HLB value of 10-15 may be particularly preferred according to the invention.

It has emerged as being further advantageous if polymers (G) are present in the compositions according to the invention. In a preferred embodiment, polymers are therefore added to the compositions according to the invention, with cationic, anionic, amphoteric, and nonionic polymers having proven to be effective.

Cationic and amphoteric polymers can preferably be used according to the invention. Cationic or amphoteric polymers are to be understood as meaning polymers which, in the main chain and/or side chain, have a group which may be “temporarily” or “permanently” cationic. According to the invention, the term “permanently cationic” is used to refer to those polymers which have a cationic group irrespective of the pH of the composition. These are generally polymers which contain a quaternary nitrogen atom, for example, in the form of an ammonium group. Preferred cationic groups are quaternary ammonium groups. In particular, those polymers in which the quaternary ammonium group is bonded via a C₁₋₄ hydrocarbon group to a polymer main chain constructed from acrylic acid, methacrylic acid or derivatives thereof have proven to be particularly suitable.

Homopolymers of the general formula (G1-I)

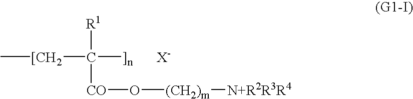

in which R¹═—H or —CH₃, R², R³ and R⁴, independently of one another, are selected from C₁₋₄-alkyl, -alkenyl or -hydroxyalkyl groups, m=1, 2, 3 or 4, n is a natural number and X⁻ is a physiologically compatible organic or inorganic anion, and copolymers consisting essentially of the monomer units listed in formula (G1-I), and nonionogenic monomer units, are particularly preferred cationic polymers. Among these polymers, preference is given according to the invention to those for which at least one of the following conditions applies:
R¹ is a methyl group,
R², R³ and R⁴ are methyl groups, or
m has the value 2.

Suitable physiologically compatible counterions X⁻ are, for example, halide ions, sulfate ions, phosphate ions, methosulfate ions, and organic ions, such as lactate, citrate, tartrate and acetate ions. Preference is given to halide ions, in particular, chloride.

A particularly suitable homopolymer is the poly(methacryloyloxyethyltrimethylammonium chloride)—crosslinked if desired—with the INCI name Polyquaternium-37. Such products are commercially available, for example, under the names Rheocare® CTH (Cosmetic Rheologies) and Synthalen® CR (Ethnichem). If desired, the crosslinking can take place with the help of polyolefinically unsaturated compounds, for example, divinylbenzene, tetraallyloxyethane, methylenebisacrylamide, diallyl ether, polyallyl polyglyceryl ether, or allyl ethers of sugars or sugar derivatives, such as erythritol, pentaerythritol, arabitol, mannitol, sorbitol, sucrose or glucose. Methylenebisacrylamide is a preferred crosslinking composition.

The homopolymer is preferably used in the form of a nonaqueous polymer dispersion which should have a polymer fraction not below 30% by weight. Such polymer dispersions are commercially available under the names Salcare® SC 95 (about 50% polymer fraction, further components: mineral oil (INCI name: Mineral Oil) and tridecyl polyoxypropylene polyoxyethylene ether (INCI name: PPG-1-Trideceth-6)) and Salcare® SC 96 (about 50% polymer fraction, further components: mixture of diesters of propylene glycol with a mixture of caprylic acid and capric acid (INCI name: Propylene Glycol Dicaprylate/Dicaprate) and tridecyl polyoxypropylene polyoxyethylene ether (INCI name: PPG-1-Trideceth-6)).

Copolymers with monomer units according to formula (G1-I) comprise, as nonionogenic monomer units, preferably acrylamide, methacrylamide, C₁₋₄-alkyl acrylates and C₁₋₄-alkyl methacrylates. Among these nonionogenic monomers, particular preference is given to acrylamide. As in the case of the homopolymers described above, these copolymers too may be crosslinked. A copolymer preferred according to the invention is the crosslinked acrylamide-methacryloyloxyethyltrimethylammonium chloride copolymer. Such copolymers in which the monomers are present in a weight ratio of about 20:80 are commercially available as about 50% strength nonaqueous polymer dispersion under the name Salcare® SC 92.

Further preferred cationic polymers are, for example,
quaternized cellulose derivatives, as are commercially available under the names Celquat® and Polymer JR®. The compounds Celquat® H 100, Celquat® L 200 and Polymer JR®400 are preferred quaternized cellulose derivatives,
cationic alkyl polyglycosides as in DE-C 44 13 686,
cationized honey, for example, the commercial product Honeyquat® 50,
cationic guar derivatives, such as, in particular, the products sold under the trade names Cosmedia® Guar and Jaguar®,
polymeric dimethyldiallylammonium salts and copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as, for example, vinylpyrrolidone-dimethylaminoethyl methacrylate copolymers quaternized with diethyl sulfate. Such compounds are commercially available under the names Gafquat® 734 and Gafquat® 755,
vinylpyrrolidone-vinylimidazolium methochloride copolymers, as are supplied under the names Luviquat® FC 370, FC 550, FC 905 and HM 552,
quaternized polyvinyl alcohol,
and the polymers known under the names Polyquaternium 2, Polyquaternium 17, Polyquaternium 18 and Polyquaternium 27 with quaternary nitrogen atoms in the polymer main chain.

As cationic polymers it is likewise possible to use the polymers known under the names Polyquaternium-24 (commercial product e.g. Quatrisoft® LM 200). According to the invention, it is likewise possible to use the copolymers of vinylpyrrolidone, as are obtainable as commercial products Copolymer 845 (manufacturer: ISP), Gaffix® VC 713 (manufacturer: ISP), Gafquat® ASCP 1011, Gafquat® HS 110, Luviquat® 8155 and Luviquat® MS 370.

Further cationic polymers which can be used in the compositions according to the invention are the so-called “temporarily cationic” polymers. These polymers usually comprise an amino group which is present as quaternary ammonium group and thus in cationic form at certain pH values. Preference is given, for example, to chitosan and derivatives thereof, as are freely available commercially, for example, under the trade names Hydagen® CMF, Hydagen® HCMF, Kytamere PC and Chitolam® NB/101.

Cationic polymers preferred according to the invention are cationic cellulose derivatives and chitosan and derivatives thereof, in particular, the commercial products Polymer® JR 400, Hydagen® HCMF and Kytame® PC, cationic guar derivatives, cationic honey derivatives, in particular, the commercial product Honeyquat® 50, cationic alkyl polyglycosides as in DE-C 44 13 686, and polymers of the Polyquaternium-37 type.

In addition, cationized protein hydrolyzates are types of cationic polymers, where the parent protein hydrolyzate can originate from animal, for example, from collagen, milk or keratin, from plant, for example, from wheat, corn, rice, potatoes, soya or almonds, from marine life forms, for example, from fish collagen or algae, or protein hydrolyzates obtained by biotechnological methods. The protein hydrolyzates on which the cationic derivatives according to the invention are based can be obtained from the corresponding proteins by a chemical, in particular, alkaline or acidic, hydrolysis, by an enzymatic hydrolysis and/or a combination of both types of hydrolysis. The hydrolysis of proteins generally gives a protein hydrolyzate with a molecular weight distribution from about 100 daltons to several thousand daltons. Preference is given here to those cationic protein hydrolyzates whose parent protein moiety has a molecular weight of from 100 to 25,000 daltons, preferably 250 to 5,000 daltons. In addition, cationic protein hydrolyzates are to be understood as meaning quaternized amino acids and mixtures thereof. The quaternization of the protein hydrolyzates or of the amino acids is often carried out using quaternary ammonium salts, such as, for example, N,N-dimethyl-N-(n-alkyl)-N-(2-hydroxy-3-chloro-n-propyl)ammonium halides. In addition, the cationic protein hydrolyzates can also be yet further derivatized. Typical examples of the cationic protein hydrolyzates and derivatives according to the invention which may be mentioned are the products specified under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702) and commercially available products: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein and Quaternium-79 Hydrolyzed Wheat Protein.

Very particular preference is given to the plant-based cationic protein hydrolyzates and derivatives.

In addition to cationic polymers, or instead of them, the compositions according to the invention can also comprise amphoteric polymers. These additionally have at least one negatively charged group in the molecule and are also referred to as zwitterionic polymers. Zwitterionic polymers which can preferably be used within the scope of the present invention are essentially composed of

A) monomers with quaternary ammonium groups of the general formula (Z-I),

R¹—CH═CR²—CO-Z-(CₙH₂ₙ)—N⁽⁺⁾R³R⁴R⁵A⁽⁻⁾ (Z-I)
in which R¹ and R², independently of one another, are hydrogen or a methyl group and R³, R⁴ and R⁵, independently of one another, are alkyl groups having 1 to 4 carbon atoms, Z is an NH group or an oxygen atom, n is an integer from 2 to 5 and A is the anion of an organic or inorganic acid and
   B) monomeric carboxylic acids of the general formula (Z-II),

R⁶—CH═CR⁷—COOH (II)
in which R⁶ and R⁷, independently of one another, are hydrogen or methyl groups.

Suitable starting monomers are, for example, dimethylaminoethylacrylamide, dimethylaminoethylmethacrylamide, dimethylaminopropylacrylamide, dimethylaminopropylmethacrylamide and diethylaminoethylacrylamide if Z is an NH group, or dimethylaminoethyl acrylate, dimethylaminoethyl methacrylate and diethylaminoethyl acrylate if Z is an oxygen atom.

The monomers containing a tertiary amino group are then quaternized in a known manner, where methyl chloride, dimethyl sulfate or diethyl sulfate are particularly suitable as alkylating reagents. The quaternization reaction can take place in aqueous solution or in the solvent.

Advantageously, monomers of the formula (Z-I) which are derivatives of acrylamide or methacrylamide are used. Preference is also given to those monomers which comprise, as counterions, halide, methoxysulfate or ethoxysulfate ions. Preference is likewise given to those monomers of the formula (Z-I) in which R³, R⁴ and R⁵ are methyl groups.

The acrylamidopropyltrimethylammonium chloride is a very particularly preferred monomer of the formula (Z-I).

Suitable monomeric carboxylic acids of the formula (Z-II) are acrylic acid, methacrylic acid, crotonic acid and 2-methylcrotonic acid. Preference is given to using acrylic or methacrylic acid, in particular, acrylic acid.

Zwitterionic polymers which can be used according to the invention are prepared from monomers of the formulae (Z-I) and (Z-II) by polymerization methods known per se. The polymerization can take place either in aqueous or aqueous-alcoholic solution. The alcohols used are alcohols having 1 to 4 carbon atoms, preferably, isopropanol, which simultaneously serve as polymerization regulators. However, other components can also be added to the monomer solution as regulator, e.g., formic acid or mercaptans, such as thioethanol and thioglycolic acid. The polymerization is initiated with the help of radical-forming substances. For this purpose, it is possible to use redox systems and/or thermally decomposing radical formers of the azo compound type, such as, for example, azoisobutyronitrile, azobis(cyanopentanoic acid) or azobis(amidinopropane) dihydrochloride. Suitable redox systems are, for example, combinations of hydrogen peroxide, potassium or ammonium peroxodisulfate, and tertiary butyl hydroperoxide with sodium sulfite, sodium dithionite or hydroxylamine hydrochloride as reduction component.

The polymerization can be carried out isothermally or under adiabatic conditions, where, depending on the concentration ratios, the temperature range for the course of the reaction can vary between 20 and 200° C. as a result of the heat of polymerization which is liberated, and the reaction, if appropriate, has to be carried out under the superatmospheric pressure which is established. Preferably, the reaction temperature is between 20 and 100° C.

The pH during the copolymerization can vary within a wide range. Advantageously, polymerization is carried out at a low pH; however, a pH above neutral is also possible. After the polymerization, an aqueous base, e.g. sodium hydroxide solution, potassium hydroxide solution or ammonia, is used to adjust the pH to between 5 and 10, preferably 6 to 8. Further details relating to the polymerization method can be found in the examples.

Polymers which have proven particularly effective are those in which the monomers of the formula (Z-I) were present in excess compared to the monomers of the formula (Z-II). It is therefore preferred according to the invention to use those polymers which consist of monomers of the formula (Z-I) and the monomers of the formula (Z-II) in a molar ratio of from 60:40 to 95:5, in particular, from 75:25 to 95:5.

The cationic and amphoteric polymers are present in the compositions according to the invention preferably in amounts of 0.05% to 10% by weight, based on the overall composition. Amounts of 0.1% to 5% by weight are particularly preferred.

The anionic polymers (G2) are anionic polymers which have carboxylate and/or sulfonate groups. Examples of anionic monomers of which such polymers can consist are acrylic acid, methacrylic acid, crotonic acid, maleic anhydride and 2-acrylamido-2-methylpropanesulfonic acid. In this connection, the acid groups can be completely or partly in the form of the sodium, potassium, ammonium, mono- or triethanolammonium salt. Preferred monomers are 2-acrylamido-2-methylpropanesulfonic acid and acrylic acid.

Anionic polymers which have proven very particularly effective are those which comprise 2-acrylamido-2-methylpropanesulfonic acid as the sole monomer or comonomer, where the sulfonic acid group may be present completely or partly in the form of the sodium, potassium, ammonium, mono- or triethanolammonium salt.

Particular preference is given to the homopolymer of 2-acrylamido-2-methyl-propanesulfonic acid, which is commercially available, for example, under the name Rheothik® 11-80.

Within this embodiment, it may be preferred to use copolymers of at least one anionic monomer and at least one nonionogenic monomer. With regard to the anionic monomers, reference is made to the substances listed above. Preferred nonionogenic monomers are acrylamide, methacrylamide, acrylic esters, methacrylic esters, vinylpyrrolidone, vinyl ethers and vinyl esters.

Preferred anionic copolymers are acrylic acid-acrylamide copolymers and, in particular, polyacrylamide copolymers with monomers containing sulfonic acid groups. A particularly preferred anionic copolymer consists of 70 to 55 mol % of acrylamide and 30 to 45 mol % of 2-acrylamido-2-methylpropanesulfonic acid, where the sulfonic acid group is present completely or partly in the form of the sodium, potassium, ammonium, mono- or triethanolammonium salt. This copolymer can also be in crosslinked form, in which case suitable crosslinking compositions are preferably polyolefinically unsaturated compounds such as tetraallyloxyethane, allyl sucrose, allyl pentaerythritol and methylenebisacrylamide. Such a polymer is present in the commercial product Sepigel 305 from SEPPIC. The use of this compound which, besides the polymer component, comprises a hydrocarbon mixture (C₁₃-C₁₄-isoparaffin) and a nonionogenic emulsifier (Laureth-7) has proven particularly advantageous within the scope of the teaching according to the invention.

The sodium acryloyldimethyltaurate copolymers sold under the name Simulgel®600 as compound with isohexadecane and polysorbate-80 have also proven to be particularly effective according to the invention.

The sodium acryloyldimethyltaurate-hydroxyethyl acrylate copolymers sold under the name Simulgel®NS as compound with squalane and polysorbate-60 have also proven to be particularly effective according to the invention.

The sodium acryloyldimethyltaurate-sodium acrylate copolymers sold under the name Simulgel®EG as compound with isohexadecane and polysorbate-80 have also proven to be particularly effective according to the invention.

The sodium acryloyldimethyltaurate-hydroxyethyl acrylate copolymers sold under the name Simulgel®EPG as compound with polyisobutene and caprylyl-/caprylglucoside have also proven to be particularly effective according to the invention.

Likewise preferred anionic homopolymers are uncrosslinked and crosslinked polyacrylic acids. Here, allyl ethers of pentaerythritol, of sucrose and of propylene may be preferred crosslinking compositions. Such compounds are commercially available, for example, under the trade name Carbopol®.

Copolymers of maleic anhydride and methyl vinyl ether, in particular, those with crosslinks, are likewise color-retaining polymers. A maleic acid-methyl vinyl ether copolymer crosslinked with 1,9-decadiene is commercially available under the name Stabileze® QM.

In addition, polymers which can be used for increasing the effect of the combination of actives according to the invention are amphoteric polymers (G3). The term “amphoteric polymers” includes both those polymers which comprise both free amino groups and also free —COOH or SO₃H groups in the molecule and are capable of forming internal salts, and also zwitterionic polymers which comprise quaternary ammonium groups and —COO⁻ or —SO₃⁻ groups in the molecule, and those polymers which comprise —COOH or SO₃H groups and quaternary ammonium groups.

One example of an amphopolymer which can be used according to the invention is the acrylic resin obtainable under the name Amphomer®, which is a copolymer of tert-butylaminoethyl methacrylate, N-(1,1,3,3-tetramethylbutyl)acrylamide and two or more monomers from the group consisting of acrylic acid, methacrylic acid and monoesters thereof.

Preferably used amphoteric polymers are those polymers which are composed essentially of

(a) monomers with quaternary ammonium groups of the general formula (G3-I),

R¹—CH═CR²—CO-Z-(CₙH₂ₙ)—N⁽⁺⁾R³R⁴R⁵A⁽⁻⁾ (G3-I)

in which R¹ and R², independently of one another, are hydrogen or a methyl group and R³, R⁴ and R⁵, independently of one another, are alkyl groups having 1 to 4 carbon atoms, Z is an NH group or an oxygen atom, n is an integer from 2 to 5 and A(−) is the anion of an organic or inorganic acid, and
(b) monomeric carboxylic acids of the general formula (G3-II),

R⁶—CH═CR⁷—COOH (G3-II)

in which R⁶ and R⁷, independently of one another, are hydrogen or methyl groups.

According to the invention, these compounds can either be used directly or in salt form, which is obtained by neutralization of the polymers, for example, with an alkali metal hydroxide. Very particular preference is given to using those polymers in which monomers of type (a) are used, in which R³, R⁴ and R⁵ are methyl groups, Z is an NH group and A(−) is a halide, methoxysulfate or ethoxysulfate ion; acrylamidopropyltrimethylammonium chloride is a particularly preferred monomer (a). The monomer (b) used for the specified polymers is preferably acrylic acid.

In a further embodiment, the compositions according to the invention can comprise nonionogenic polymers (G4).

Suitable nonionogenic polymers are, for example:
vinylpyrrolidone/vinyl ester copolymers, as are sold, for example, under the trade name Luviskol® (BASF). Luviskol® VA 64 and Luviskol® VA 73, in each case vinylpyrrolidone/vinyl acetate copolymers, are likewise preferred nonionic polymers.
cellulose ethers, such as hydroxypropylcellulose, hydroxyethylcellulose and methyl-hydroxypropylcellulose, as are sold, for example, under the trade name Culminal® and Benecel® (AQUALON) and Natrosol® grades (Hercules)
starch and derivatives thereof, in particular, starch ethers, for example, Structure® XL (National Starch), a multifunctional, salt-tolerant starch;
shellac
polyvinylpyrrolidones, as are sold, for example, under the name Luviskol® (BASF).
siloxanes. These siloxanes may either be water-soluble or water-insoluble. Of suitability are both volatile and nonvolatile siloxanes, where nonvolatile siloxanes are understood as meaning those compounds whose boiling point at atmospheric pressure is above 200° C. Preferred siloxanes are polydialkylsiloxanes, such as, for example, polydimethylsiloxane, polyalkylarylsiloxanes, such as, for example, polyphenylmethylsiloxane, ethoxylated polydialkylsiloxanes, and polydialkylsiloxanes which contain amine and/or hydroxy groups.
glycosidically substituted silicones.

According to the invention, it is also possible for the preparations used to comprise a plurality of, in particular, two, different polymers of identical charge and/or in each case one ionic and one amphoteric and/or nonionic polymer.

The polymers (G) are present in the compositions according to the invention preferably in amounts of 0.05% to 10% by weight, based on the overall composition. Amounts of 0.1% to 5% by weight, in particular, of 0.1% to 3% by weight, are particularly preferred.

In addition to the specified substances, the compositions according to the invention can comprise further care substances. These are particularly advantageously, for example, vitamins, provitamins or vitamin precursors, meaning that compositions preferred according to the invention are characterized in that they further comprise at least one substance from the group of vitamins, provitamins and vitamin precursors, and derivatives thereof, preference being given to vitamins, provitamins and vitamin precursors which are assigned to the groups A, B, E, F and H. These have been described in detail above.

A further group of care substances which may be present in the compositions according to the invention are the protein hydrolyzates and derivatives thereof (P). Protein hydrolyzates are product mixtures which are obtained by acidically, basically or enzymatically catalyzed degradation of proteins. According to the invention, the term “protein hydrolyzates” is also understood as meaning total hydrolyzates, and individual amino acids and derivatives thereof, and mixtures of different amino acids. Furthermore, according to the invention, polymers constructed from amino acids and amino acid derivatives are covered by the term protein hydrolyzates. The latter include, for example, polyalanine, polyasparagine, polyserine etc. Further examples of compounds which can be used according to the invention are L-alanyl-L-proline, polyglycine, glycyl-L-glutamine or D/L-methionine-5-methylsulfonium chloride. According to the invention, it is of course also possible to use β-aminoacids and derivatives thereof, such as β-alanine, anthranilic acid or hippuric acid. The molar weight of the protein hydrolyzates which can be used according to the invention is between 75, the molar weight of glycine, and 200,000; preferably, the molar weight is 75 to 50,000 and very particularly preferably 75 to 20,000 daltons.

According to the invention, protein hydrolyzates both of vegetable and animal or marine or synthetic origin may be used.

Animal protein hydrolyzates are, for example, elastin, collagen, keratin and milk protein hydrolyzates, which may also be in the form of salts. Such products are sold, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

According to the invention, preference is given to the use of protein hydrolyzates of vegetable origin, e.g., soya, almond, pea, potato and wheat protein hydrolyzates. Such products are available, for example, under the trade names Gluadin® (Cognis), DiaMin® (Dia-malt), Lexein® (Inolex), Hydrosoy® (Croda), Hydrolupin® (Croda), Hydrosesame® (Croda), Hydrotritium® (Croda) and Crotein® (Croda).

Although the use of the protein hydrolyzates as such is preferred, it is also possible, if appropriate, to use instead amino acid mixtures obtained in another way. The use of derivatives of the protein hydrolyzates, for example, in the form of their fatty acid condensation products, is likewise possible. Such products are sold, for example, under the names Lamepon® (Cognis), Lexein® (Inolex), Crolastin® (Croda) or Crotein® (Croda).

The teaching according to the invention of course includes all isomeric forms, such as cis—, transisomers, diastereomers and chiral isomers.

According to the invention, it is also preferable to use a mixture of two or more protein hydrolyzates (P).

The protein hydrolyzates (P) are present in the compositions preferably, in concentrations of from 0.01% by weight to 20% by weight, more preferably, from 0.05% by weight to 15% by weight and very particularly preferably, in amounts of from 0.05% by weight to 5% by weight.

The compositions according to the invention can also comprise preferably a 2-pyrrolidinone-5-carboxylic acid and derivatives thereof (J). Particular preference is given to the sodium, potassium, calcium, magnesium or ammonium salts in which the ammonium ion carries one to three C₁- to C₄-alkyl groups besides hydrogen. The sodium salt is very particularly preferred. The amounts used in the compositions according to the invention are preferably, 0.05% to 10% by weight, based on the overall composition, particularly preferably, 0.1% to 5% by weight, and in particular, 0.1% to 3% by weight.

Finally, in one preferred embodiment, the compositions according to the invention can also comprise plant extracts (L).

These extracts are usually prepared by extracting the whole plant. However, in individual cases, it may also be preferred to prepare the extracts exclusively from flowers and/or leaves of the plant.

With regard to the plant extracts which can be used according to the invention, reference is made, in particular, to the extracts which are listed in the table starting on page 44 of the 3rd edition of the Leitfadens zur Inhaltestoffdeklaration kosmetischer Mittel [Introduction to the ingredients declaration of cosmetic compositions], published by the Industrieverband Körperpflege—und Waschmittel e.V. (IKW), Frankfurt.

According to the invention, the extracts from green tea, oak bark, stinging nettle, hamamelis, hops, henna, camomile, burdock root, horsetail, hawthorn, linden blossom, almond, aloe vera, spruce needle, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root in particular, are preferred.

Extractants for producing the specified plant extracts which may be used are water, alcohols and mixtures thereof. Among the alcohols, preference is given here to lower alcohols, such as ethanol and isopropanol, but in particular, polyhydric alcohols, such as ethylene glycol and propylene glycol, both as the sole extractant and also in a mixture with water. Plant extracts based on water/propylene glycol in the ratio 1:10 to 10:1 have proven to be particularly suitable.

According to the invention, the plant extracts can be used either in pure form or in dilute form. If they are used in dilute form, they usually comprise about 2%-80% by weight of active substance and, as solvent, the extractant or extractants mixture used during their isolation.

In addition, it may be preferred to use mixtures of two or more, in particular, of two, different plant extracts in the compositions according to the invention.

In addition, it may prove advantageous if penetrants and/or swellants (M) are present in the compositions according to the invention. These include, for example, urea and urea derivatives, guanidine and derivatives thereof, arginine and derivatives thereof, waterglass, imidazole and derivatives thereof, histidine and derivatives thereof, benzyl alcohol, glycerol, glycol and glycol ethers, propylene glycol and propylene glycol ethers, for example, propylene glycol monoethyl ethers, carbonates, hydrogen carbonates, diols and triols, and, in particular, 1,2-diols and 1,3-diols, such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol.

Advantageously, for the purposes of the invention, short-chain carboxylic acids (N) can additionally assist the inventive combination of actives. For the purposes of the invention, short-chain carboxylic acids and derivatives thereof are understood as meaning carboxylic acids which may be saturated or unsaturated and/or straight-chain or branched or cyclic and/or aromatic and/or heterocyclic and have a molecular weight of less than 750. For the purposes of the invention, saturated or unsaturated straight-chain or branched carboxylic acids with a chain length of from 1 to 16 carbon atoms in the chain may be preferred, very particular preference being given to those with a chain length of from 1 to 12 carbon atoms in the chain.

For the purposes of the invention, the short-chain carboxylic acids can have one, two, three or more carboxy groups. For the purposes of the invention, preference is given to carboxylic acids with two or more carboxy groups, in particular, di- and tricarboxylic acids. The carboxy groups may be present completely or in part as ester, acid anhydride, lactone, amide, imidic acid, lactam, lactim, dicarboximide, carbohydrazide, hydrazone, hydroxam, hydroxime, amidine, amide oxime, nitrile, phosphonic or phosphate ester. The carboxylic acids according to the invention can, of course, be substituted along the carbon chain or the ring backbone. The substituents of the preferred additional carboxylic acids according to the invention are to include, for example, C1-C8-alkyl, C2-C8-alkenyl, aryl, aralkyl and aralkenyl, hydroxymethyl, C2-C8-hydroxyalkyl, C2-C8-hydroxyalkenyl, aminomethyl, C2-C8-aminoalkyl, cyano, formyl, oxo, thioxo, hydroxyl, mercapto, amino, carboxy or imino groups. Preferred substituents are C1-C8-alkyl, hydroxymethyl, hydroxyl, amino and carboxy groups. Particular preference is given to substituents in the a position. Very particularly preferred substituents are hydroxyl, alkoxy and amino groups, where the amino function may optionally be further substituted by alkyl, aryl, aralkyl and/or alkenyl radicals. Furthermore, likewise preferred carboxylic acid derivatives are the phosphonic and phosphate esters.

Examples of carboxylic acids according to the invention which may be mentioned are formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluoyl acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehydic acid, 4-hydroxyphthalamic acid, 1-pyrazolecarboxylic acid, gallic acid or propanetricarboxylic acid, a dicarboxylic acid chosen from the group which is formed by compounds of the general formula (N-I),

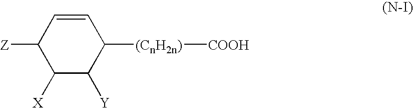

in which Z is a linear or branched alkyl or alkenyl group having 4 to 12 carbon atoms, n is a number from 4 to 12, and one of the two groups X and Y is a COOH group and the other is hydrogen or a methyl or ethyl radical, dicarboxylic acids of the general formula (N-I) which additionally also carry 1 to 3 methyl or ethyl substituents on the cyclohexene ring, and dicarboxylic acids which form from the dicarboxylic acids according to formula (N-I) formally by addition of a molecule of water onto the double bond in the cyclohexene ring.

Dicarboxylic acids of the formula (N-I) are known in the literature.

The dicarboxylic acids of the formula (N-I) can be prepared, for example, by reacting polyunsaturated dicarboxylic acids with unsaturated monocarboxylic acids in the form of a Diels-Alder cyclization. The process usually starts from a polyunsaturated fatty acid as dicarboxylic acid component. Preference is given to the linoleic acid obtainable from natural fats and oils. As monocarboxylic acid component, preference is given in particular, to acrylic acid, but also, for example, methacrylic acid and crotonic acid. Usually, in reactions according to Diels-Alder, isomer mixtures are formed in which one component is present in excess. According to the invention, these isomer mixtures can be used just as much as the pure compounds.

Besides the preferred dicarboxylic acids according to formula (N-I), according to the invention it is also possible to use those dicarboxylic acids which differ from the compounds according to formula (N-I) by 1 to 3 methyl or ethyl substituents on the cyclohexyl ring or are formed from these compounds formally by adding a molecule of water onto the double bond of the cyclohexene ring.

The dicarboxylic acid (mixture) which forms by reacting linoleic acid with acrylic acid has proven particularly effective according to the invention. This is a mixture of 5- and 6-carboxy-4-hexyl-2-cyclohexene-1-octanoic acid. Such compounds are commercially available under the names Westvaco Diacid® 1550 and Westvaco Diacid® 1595 (manufacturer: Westvaco).

Besides the short-chain carboxylic acids according to the invention themselves listed above by way of example as additionally preferred, it is also possible to use their physiologically compatible salts according to the invention. Examples of such salts are the alkali metal salts, alkaline earth metal salts, zinc salts and also ammonium salts, which, for the purposes of the present application, are also understood as meaning the mono-, di- and trimethyl-, -ethyl- and -hydroxyethylammonium salts. However, for the purposes of the invention, very particular preference may be given to using acids neutralized with alkaline-reacting amino acids, such as, for example, arginine, lysine, ornithine and histidine. Furthermore, it may be preferred, for formulation reasons, to choose the carboxylic acid from the water-soluble representatives, in particular, the water-soluble salts.

In addition, it is preferred according to the invention to use hydroxycarboxylic acids and here in turn, in particular, the dihydroxy-, trihydroxy- and polyhydroxycarboxylic acids, and the dihydroxy-, trihydroxy- and polyhydroxy- di-, tri- and polycarboxylic acids together with the active ingredient combination of the invention. In this connection, it has been found that, besides the hydroxycarboxylic acids, the hydroxycarboxylic acid esters, and also the mixtures of hydroxycarboxylic acids and esters thereof, and also polymeric hydroxycarboxylic acids and esters thereof may also be very particularly preferred. Preferred hydroxycarboxylic acid esters are, for example, full esters of glycolic acid, lactic acid, malic acid, tartaric acid or citric acid. Further fundamentally suitable hydroxycarboxylic acid esters are esters of β-hydroxypropionic acid, of tartronic acid, of D-gluconic acid, of sugar acid, of mucic acid or of glucuronic acid. Suitable as alcohol components of these esters are primary, linear or branched aliphatic alcohols having 8-22 carbon atoms, thus, for example, fatty alcohols or synthetic fatty alcohols. Here, the esters of C12-C15 fatty alcohols are particularly preferred. Esters of this type are commercially available, e.g., under the trade name Cosmacol® from EniChem, Augusta Industriale. Particularly preferred polyhydroxypolycarboxylic acids are polylactic acid and polytartaric acid, and esters thereof.

Cosmetic or dermatological preparations which are further preferred in accordance with the invention are characterized in that they further contain 0.5% to 2.5% by weight, preferably 0.75% to 1.5% by weight of at least one of the following listed actives/preparations of actives as it is:

| | | Supplier/ |
|---|---|---|
| Trade Name | INCI | Manufacturer |
| Polyol Licorice | GLYCYRRHIZA GLABRA | Jan Dekker/ |
| P-T 40 | | Nikko |
| Aqua Licorice | GLYCYRRHIZA GLABRA, | Jan Dekker/ |
| Extract P-T | PPG-6- | Nikko |
| | DECYLTETRADECETH-30, | |
| | BUTYLENE GLYCOL, | |
| | WATER | |
| Melaslow | CITRUS UNSHIU | Sederma |
| | (PEEL EXTRACT), | |
| | GLYCERIN | |
| Phyliance | PHYTIC ACID | IMPAG |
| Lumiskin | CAPRYLIC/CAPRIC | Sederma |
| | TRIGLYCERIDE (AND) | |
| | DIACETYLBOLDINE | |
| Melanostatine DM | WATER, | IMPAG |
| | DEXTRAN | |
| | (AND) HEXAPEPTIDE-2, | |
| | PHENONIP | |

Cosmetic or dermatological preparations that are particularly preferred in accordance with the invention further comprise silicone(s). Particularly preferred cosmetic or dermatological preparations of the invention here are characterized in that they further contain silicone(s), preferably in amounts of 0.1% to 10% by weight, preferably of 0.25% to 7% by weight, and more particularly of 0.5% to 5% by weight, based in each case on the overall composition.

Particular preference is given to cosmetic or dermatological preparations according to the invention which comprise at least one silicone selected from:
(i) polyalkylsiloxanes, polyarylsiloxanes, polyalkylarylsiloxanes, which are volatile or nonvolatile, straight-chain, branched or cyclic, crosslinked or uncrosslinked;
(ii) polysiloxanes which contain one or more organofunctional groups in their general structure which are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane(A)-polyoxyalkylene(B) block copolymers of the type (A-B)ₙ where n>3;
(iv) grafted silicone polymers with a non-silicone-containing, organic backbone, which consist of an organic main chain which is formed from organic monomers which do not contain silicone, onto which at least one polysiloxane macromer has been grafted in the chain and optionally on at least one chain end;
(v) grafted silicone polymers with polysiloxane backbone, onto which non-silicone containing, organic monomers have been grafted which have a polysiloxane main chain, onto which at least one organic macromer which does not contain silicone has been grafted in the chain and optionally on at least one of its ends;
(vi) or mixtures thereof.

Particularly preferred cosmetic or dermatological preparations according to the invention are characterized in that they comprise at least one silicone of the formula I

(CH₃)₃Si—[O—Si(CH₃)₂]ₓ—O—Si(CH₃)₃ (I),

in which x is a number from 0 to 100, preferably from 0 to 50, more preferably from 0 to 20, and particularly 0 to 10.

The cosmetic or dermatological preparations preferred according to the invention comprise a silicone of the above formula I. These silicones are referred to as DIMETHICONE according to INCI nomenclature. For the purposes of the present invention, the silicone of the formula I used is preferably the compounds:

(CH₃)₃Si—O—Si(CH₃)₃

(CH₃)₃Si—O—(CH₃)₂Si—O—Si(CH₃)₃

(CH₃)₃Si—[O—(CH₃)₂Si]₂—O—Si(CH₃)₃

(CH₃)₃Si—[O—(CH₃)₂Si]₃—O—Si(CH₃)₃

(CH₃)₃Si—[O—(CH₃)₂Si]₄—O—Si(CH₃)₃

(CH₃)₃Si—[O—(CH₃)₂Si]₅—O—Si(CH₃)₃

(CH₃)₃Si—[O—(CH₃)₂Si]₆—O—Si(CH₃)₃

(CH₃)₃Si—[O—(CH₃)₂Si]₇—O—Si(CH₃)₃

(CH₃)₃Si—[O—(CH₃)₂Si]₈—O—Si(CH₃)₃

(CH₃)₃Si—[O—(CH₃)₂Si]₉—O—Si(CH₃)₃

(CH₃)₃Si—[O—(CH₃)₂Si]₁₀—O—Si(CH₃)₃

(CH₃)₃Si—[O—(CH₃)₂Si]₁₁—O—Si(CH₃)₃

(CH₃)₃Si—[O—(CH₃)₂Si]₁₂—O—Si(CH₃)₃

(CH₃)₃Si—[O—(CH₃)₂Si]₁₃—O—Si(CH₃)₃

(CH₃)₃Si—[O—(CH₃)₂Si]₁₄—O—Si(CH₃)₃

(CH₃)₃Si—[O—(CH₃)₂Si]₁₅—O—Si(CH₃)₃

(CH₃)₃Si—[O—(CH₃)₂Si]₁₆—O—Si(CH₃)₃

(CH₃)₃Si—[O—(CH₃)₂Si]₁₇—O—Si(CH₃)₃

(CH₃)₃Si—[O—(CH₃)₂Si]₁₈—O—Si(CH₃)₃

(CH₃)₃Si—[O—(CH₃)₂Si]₁₉—O—Si(CH₃)₃

(CH₃)₃Si—[O—(CH₃)₂Si]₂₀—O—Si(CH₃)₃

where (CH₃)₃Si—O—Si(CH₃)₃, (CH₃)₃Si—O—(CH₃)₂Si—O—Si(CH₃)₃ and/or (CH₃)₃Si—[O—(CH₃)₂Si]₂-β—Si(CH₃)₃ are particularly preferred.

Mixtures of the above-mentioned silicones may of course also be present in the preferred compositions according to the invention.

Preferred silicones which can be used according to the invention have, at 20° C., viscosities of from 0.2 to 2 mm²s⁻¹, where silicones with viscosities of from 0.5 to 1 mm²s⁻¹ are particularly preferred.

Particularly preferred compositions according to the invention comprise one or more amino-functional silicones. Such silicones can, for example, be described by the formula

M(RₐQ_{b}SiO_{(4-a-b)/2)x}(R_{c}SiO_{(4-c)/2)y}M

where, in the above formula, R is a hydrocarbon or a hydrocarbon radical having 1 to about 6 carbon atoms, Q is a polar radical of the general formula —R¹HZ, in which R¹ is a divalent, linking group which is bonded to hydrogen and the radical Z, composed of carbon and hydrogen atoms, carbon, hydrogen and oxygen atoms or carbon, hydrogen and nitrogen atoms, and Z is an organic, amino-functional radical which contains at least one aminofunctional group; “a” assumes values in the range from about 0 to about 2, “b” assumes values in the range from about 1 to about 3, “a”+“b” is less than or equal to 3, and “c” is a number in the range from about 1 to about 3, and x is a number in the range from 1 to about 2,000, preferably from about 3 to about 50 and most preferably from about 3 to about 25, and y is a number in the range from about 20 to about 10,000, preferably from about 125 to about 10,000 and most preferably from about 150 to about 1,000, and M is a suitable silicone end group, as is known in the prior art, preferably trimethylsiloxy. Nonlimiting examples of the radicals represented by R include alkyl radicals, such as methyl, ethyl, propyl, isopropyl, butyl, isobutyl, amyl, isoamyl, hexyl, isohexyl and the like; alkenyl radicals, such as vinyl, halovinyl, alkylvinyl, allyl, haloallyl, alkylallyl; cycloalkyl radicals, such as cyclobutyl, cyclopentyl, cyclohexyl and the like; phenyl radicals, benzyl radicals, halogenated hydrocarbon radicals, such as 3-chloropropyl, 4-bromobutyl, 3,3,3-trifluoropropyl, chlorocyclohexyl, bromophenyl, chlorophenyl and the like, and sulfur-containing radicals, such as mercaptoethyl, mercaptopropyl, mercaptohexyl, mercaptophenyl and the like; preferably, R is an alkyl radical which contains 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic amino-functional radical comprising at least one functional amino group. One possible formula for Z is NH(CH₂)_{z}NH₂, in which z is 1 or more. Another possible formula for Z is —NH(CH₂)_{z}(CH₂)zzNH, in which both z and zz, independently, are 1 or more, where this structure includes diamino ring structures, such as piperazinyl. Z is most preferably a —NHCH₂CH₂NH₂ radical. Another possible formula for Z is —N(CH₂)_{z}(CH₂)zzNX₂ or —NX₂, in which each X is selected independently of X₂ from the group consisting of hydrogen and alkyl groups having 1 to 12 carbon atoms, and zz is 0.

Q is most preferably a polar, amino-functional radical of the formula —CH₂CH₂CH₂NHCH₂CH₂NH₂. In the formulae, “a” assumes values in the range from about 0 to about 2, “b” assumes values in the range from about 2 to about 3, “a”+“b” is less than or equal to 3, and “c” is a number in the range from about 1 to about 3. The molar ratio of RₐQ_{b}SiO_{(4-a-b)/2} units to the R_{c}SiO_{(4-c)/2} units is in the range from about 1:2 to 1:65, preferably from about 1:5 to about 1:65 and most preferably from about 1:15 to about 1:20. If one or more silicones of the above formula are used, then the various variable substituents in the above formula can be different for the various silicone components which are present in the silicone mixture.

Preferred cosmetic or dermatological preparations according to the invention comprise an aminofunctional silicone of the formula (II)

R′ₐG₃₋ₐ-Si(OSiG₂)ₙ—(OSiG_{b}R′_{2-b})ₘ—O—SiG₃₋ₐ-R′ₐ (II),

in which:
G is —H, a phenyl group, —OH, —O—CH₃, —CH₃, —O—CH₂CH₃, CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a is a number between 0 and 3, more particularly 0;
b is a number between 0 and 1, more particularly 1,
m and n are numbers whose sum (m+n) is between 1 and 2,000, preferably between 50 and 150, with n preferably adopting values from 0 to 1,999 and more particularly from 49 to 149, and m preferably adopting values from 1 to 2,000, more particularly from 1 to 10,
R′ is a monovalent radical selected from
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻,
      where each Q is a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
      R″ stands for identical or different radicals from the group —H, -phenyl, -benzyl, —CH₂—CH(CH₃)Ph, the C₁₋₂₀-alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion which is preferably selected from chloride, bromide, iodide or methosulfate.

Particularly preferred cosmetic or dermatological preparations according to the invention are characterized in that they comprise at least one amino-functional silicone of the formula (IIa)

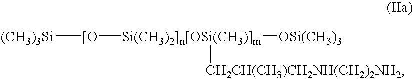

in which m and n are numbers whose sum (m+n) is between 1 and 2,000, preferably between 50 and 150, where n preferably assumes values of from 0 to 1,999 and in particular, from 49 to 149 and m preferably assumes values of from 1 to 2,000, in particular, from 1 to 10.

These silicones are referred to as trimethylsilylamodimethicones according to the INCI declaration.

Particular preference is also given to cosmetic or dermatological preparations according to the invention which comprise at least one amino-functional silicone of the formula (IIb)

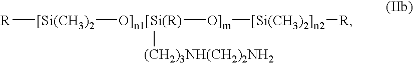

in which R is —OH, —O—CH₃ or a —CH₃ group, and m, n1 and n2 are numbers whose sum (m+n1+n2) is between 1 and 2,000, preferably between 50 and 150, where the sum (n1+n2) preferably assumes values from 0 to 1,999 and in particular, from 49 to 149 and m preferably assumes values of from 1 to 2,000, in particular, from 1 to 10.

These silicones are referred to as amodimethicones according to the INCI declaration.

Irrespective of which amino-functional silicones are used, preference is given to cosmetic or dermatological preparations according to the invention which comprise an amino-functional silicone whose amine number is above 0.25 meq/g, preferably above 0.3 meq/g and in particular, above 0.4 meq/g. The amine number here is the milli-equivalents of amine per gram of amino-functional silicone. It can be determined by titration and also quoted in the unit mg KOH/g.

Cosmetic or dermatological preparations preferred according to the invention are characterized in that they contain, based on their weight, 0.01% to 10% by weight, preferably 0.1% to 8% by weight, more preferably 0.25% to 7.5% by weight, and more particularly 0.5% to 5% by weight of amino-functional silicone(s).

Cyclic dimethicones referred to in accordance with INCI as CYCLOMETHICONE can also advantageously be used according to the invention. Here, preference is given to cosmetic or dermatological preparations according to the invention which comprise at least one silicone of the formula III

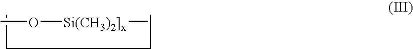

in which x is a number from 0 to 200, preferably from 0 to 10, more preferably from 0 to 7, and more particularly 0, 1, 2, 3, 4, 5 or 6.

The silicones described above have a backbone which is constructed from —Si—O—Si-units. These Si—O—Si units can of course also be interrupted by carbon chains. Corresponding molecules are accessible by chain-extension reactions and are preferably used in the form of silicone-in-water emulsions.

The silicone-in-water emulsions which can be used according to the invention can be produced by known methods, as are disclosed, for example, in U.S. Pat. No. 5,998,537 and EP 0 874 017 A1.

In summary, this production method involves the emulsifying mixing of components, one of which comprises at least one polysiloxane, the other of which comprises at least one organosilicone material which reacts with the polysiloxane in a chain-extension reaction, where at least one metal-ion-containing catalyst for the chain-extension reaction, at least one surfactant and water are present.

Chain-extension reactions with polysiloxanes are known and can involve, for example, the hydrosilylation reaction in which an Si—H group reacts with an aliphatically unsaturated group in the presence of a platinum/rhodium catalyst to form polysiloxanes with some Si—(C)ₚ—Si bonds (p=1-6), where the polysiloxanes are also referred to as polysiloxane-polysilalkylene copolymers.

The chain-extension reaction can also involve the reaction of an Si—OH group (for example, a hydroxy-terminated polysiloxane) with an alkoxy group (for example, alkoxysilanes, silicates or alkoxysiloxanes) in the presence of a metal-containing catalyst to form polysiloxanes.

The polysiloxanes which are used in the chain-extension reaction include a substantially linear polymer of the following structure:

R—Si(R₂)—[—O—Si(R₂)—]ₙ—O—SiR₃

In this structure, each R, independently of the others, is a hydrocarbon radical having up to 20 carbon atoms, preferably, having 1 to 6 carbon atoms, such as, for example, an alkyl group (for example, methyl, ethyl, propyl or butyl), an aryl group (for example, phenyl), or the group required for the chain-extension reaction (“reactive group”, for example, Si-bonded H atoms, aliphatically unsaturated groups, such as vinyl, allyl or hexenyl, hydroxy, alkoxy, such as methoxy, ethoxy or propoxy, alkoxy-alkoxy, acetoxy, amino etc.), with the proviso, that on average, one to two reactive groups are present per polymer, and n is a positive number >1. Preferably, a majority of the reactive groups, particularly preferably >90%, and in particular, >98%, of the reactive groups is bonded to the terminal Si atoms in the siloxane. Preferably, n is numbers which describe polysiloxanes which have viscosities between 1 and 1,000,000 mm²/s, particularly preferably viscosities between 1,000 and 100,000 mm²/s.

The polysiloxanes can be branched to a slight degree (for example, <2 mol % of the siloxane units), but the polymers are substantially linear, particularly preferably, completely linear. Furthermore, the substituents R can in turn be substituted, for example, by N-containing groups (for example, amino groups), epoxy groups, S-containing groups, Si-containing groups, O-containing groups etc. Preferably, at least 80% of the radicals R are alkyl radicals, particularly, preferably methyl groups.

The organosilicone material which reacts with the polysiloxane in the chain-extension reaction can either be a second polysiloxane or a molecule which acts as chain extender. If the organosilicone material is a polysiloxane, it has the general structure mentioned above. In these cases, a polysiloxane in the reaction has (at least) one reactive group, and a second polysiloxane has (at least) a second reactive group which reacts with the first group.

If the organosilicone material comprises a chain-extension agent, this may be one material, such as, for example, a silane, a siloxane (for example, disiloxanes or trisiloxane) or a silazane. Thus, for example, a composition which comprises a polysiloxane according to the general structure described above which has at least one Si—OH group can be chain-extended by reacting it with an alkoxysilane (for example, a dialkoxysilane or trialkoxysilane) in the presence of tin- or titanium-containing catalysts.

The metal-containing catalysts in the chain-extension reaction are mostly specific for a certain reaction. Such catalysts are known in the prior art and comprise, for example, metals, such as platinum, rhodium, tin, titanium, copper, lead, etc. In a preferred chain-extension reaction, a polysiloxane with at least one aliphatically unsaturated group, preferably an end group, is reacted with an organosilicone material in the presence of a hydrosilylation catalyst which is a siloxane or polysiloxane with at least one (preferably terminal) Si—H group. The polysiloxane has at least one aliphatically unsaturated group and satisfies the general formula given above in which R and n are as defined above, where, on average, between 1 and 2 groups R have one aliphatically unsaturated group per polymer. Representative aliphatically unsaturated groups are, for example, vinyl, allyl, hexenyl and cyclohexenyl or a group R²CH═CHR³, in which R² is a divalent aliphatic chain bonded to the silicon and R³ is a hydrogen atom or an alkyl group. The organosilicone material with at least one Si—H group preferably has the above-mentioned structure in which R and n are as defined above and where, on average, between 1 and 2 groups R are a hydrogen and n is 0 or a positive integer.

This material can be a polymer or a material with low molecular weight such as a siloxane (for example, a disiloxane or a trisiloxane).

The polysiloxane having at least one aliphatically unsaturated group and the organosilicone material having at least one Si—H group react in the presence of a hydrosilylation catalyst. Such catalysts are known from the prior art and include, for example, platinum- and rhodium-containing materials. The catalysts can assume any known form, for example, platinum or rhodium applied to support materials (such as, for example, silica gel or activated carbon), or other suitable compounds, such as platinum chloride, salts of platinic or chloroplatinic acids. A catalyst preferred on account of the good dispersibility in organosilicone systems and the slight color change is chloroplatinic acid either in the form of the commercially available hexahydrate or in anhydrous form.

In a further preferred chain-extension reaction, a polysiloxane having at least one Si—OH group, preferably, an end group, is reacted with an organosilicone material which has at least one alkoxy group, preferably, a siloxane having at least one Si—OR group or an alkoxy silane having at least two alkoxy groups. Here, the catalyst used is again a metal-containing catalyst.

For the reaction of an Si—OH group with an Si—OR group there are many catalysts known in the literature, for example, organometallic compounds, such as organotin salts, titanates or titanium chelates and complexes. Examples include tin octoate, dibutyltin dilaurate, dibutyltin diacetate, dimethyltin dineodecanoate, dibutyltin dimethoxide, isobutyltin triceroate, dimethyltin dibutyrate, dimethyltin dineodecanoate, triethyltin tartrate, tin oleate, tin naphthenate, tin butyrate, tin acetate, tin benzoate, tin sebacate, tin succinate, tetrabutyl titanate, tetraisopropyl titanate, tetraphenyl titanate, tetraoctadecyl titanate, titanium naphthanate, ethyltriethanolamine titanate, titanium diisopropyldiethylacetoacetate, titanium diisopropoxydiacetylacetonate and titanium tetraalkoxides in which the alkoxide is butoxy or propoxy.

The silicone-in-water emulsions moreover preferably comprise at least one surfactant.

Cosmetic or dermatological preparations likewise preferred according to the invention are characterized in that they comprise at least one silicone of the formula IV

R₃Si—[O—Si R₂]X—(CH₂)ₙ—[O—Si R₂]_{y}—O—Si R₃ (IV),

in which R stands for identical or different radicals from the group —H, -phenyl, -benzyl, —CH₂—CH(CH₃)Ph, the C120- alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y are each a number from 0 to 200, preferably, from 0 to 10, more preferably, from 0 to 7, and more particularly, 0, 1, 2, 3, 4, 5 or 6, and n is a number from 0 to 10, preferably, from 1 to 8, and more particularly is 2, 3, 4, 5 or 6.

The silicones are preferably water-soluble. Cosmetic or dermatological preparations preferred according to the invention are characterized in that they additionally comprise a water-soluble silicone.

The compositions of the invention may comprise perfumes, perfume oils or perfume-oil constituents. In the context of the present invention, perfume oils and/or fragrances used may be individual odorant compounds, for example, the synthetic products of the ester, ether, aldehyde, ketone, alcohol and hydrocarbon type. Odorant compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethyl methyl phenylglycinate, allyl cyclohexylpropionate, styrallyl propionate, benzyl salicylate, cyclohexyl salicylate, floramate, melusate, and jasmecyclate. The ethers include, for example, benzyl ethyl ether and ambroxane; the aldehydes include, for example, the linear alkanals having 8-18 carbon atoms, citral, citronellal, citronellyloxyacetaldehyde, cyclamen aldehyde, lilial and bourgeonal; the ketones include, for example, the ionones, α-isomethylionone and methyl cedryl ketone; the alcohols include anethole, citronellol, eugenol, geraniol, linalool, phenylethyl alcohol and terpineol; the hydrocarbons include primarily the terpenes such as limonene and pinene. However, preference is given to using mixtures of different odorants which together produce a pleasing fragrance note.

Such perfume oils may also comprise natural odorant mixtures, as are obtainable from vegetable sources, for example, pine oil, citrus oil, jasmine oil, patchouli oil, rose oil or ylang-ylang oil. Likewise suitable are clary sage oil, camomile oil, clove oil, balm oil, mint oil, cinnamon leaf oil, lime blossom oil, juniper berry oil, vetiver oil, olibanum oil, galbanum oil and labdanum oil, and also orange blossom oil, neroli oil, orange peel oil and sandalwood oil.

In order to be perceptible, an odorant must be volatile, for which an important role is played not only by the nature of the functional groups and by the structure of the chemical compound but also by the molar mass. Thus, the majority of odorants have molar masses of up to about 200 daltons, while molar masses of 300 daltons or more tend to be an exception. On the basis of the different volatility of odorants there is a change in the odor of a perfume or fragrance composed of two or more odorants during its evaporation, and the perceived odors are divided into top note, heart note or middle note or body, and end note or dryout. Since the perception of odor is to a large extent also based on the odor intensity, the top note of a perfume or fragrance does not consist only of volatile compounds, whereas the end note consists for the most part of less volatile odorants, i.e., odorants which adhere firmly. In the composition of perfumes it is possible for more volatile odorants, for example, to be bound to certain fixatives, which prevent them from evaporating too rapidly. The subsequent classification of the odorants into “more volatile” and “firmly adhering” odorants, therefore, states nothing about the perceived odor and about whether the odorant in question is perceived as a top note or as a heart note.

Examples of firmly adhering odorants which can be used in the context of the present invention are the essential oils such as angelica root oil, aniseed oil, arnica blossom oil, basil oil, bay oil, bergamot oil, champaca blossom oil, noble fir oil, noble fir cone oil, elemi oil, eucalyptus oil, fennel oil, spruce needle oil, galbanum oil, geranium oil, ginger grass oil, guaiacwood oil, gurjun balsam oil, helichrysum oil, ho oil, ginger oil, iris oil, cajeput oil, calamus oil, camomile oil, camphor oil, canaga oil, cardamom oil, cassia oil, pine needle oil, copaiva balsam oil, coriander oil, spearmint oil, caraway oil, cumin oil, lavender oil, lemon grass oil, lime oil, mandarin oil, balm oil, musk seed oil, myrrh oil, clove oil, neroli oil, niaouli oil, olibanum oil, orange oil, origanum oil, palmarosa oil, patchouli oil, peru balsam oil, petitgrain oil, pepper oil, peppermint oil, pimento oil, pine oil, rose oil, rosemary oil, sandalwood oil, celery oil, spike oil, star anise oil, turpentine oil, thuja oil, thyme oil, verbena oil, vetiver oil, juniperberry oil, wormwood oil, wintergreen oil, ylang-ylang oil, hyssop oil, cinnamon oil, cinnamon leaf oil, citronella oil, lemon oil and cypress oil.

However, the higher-boiling or solid odorants of natural or synthetic origin may also be used in the context of the present invention as firmly adhering odorants or odorant mixtures, i.e., fragrances. These compounds include the following compounds and mixtures thereof: ambrettolide, α-amylcinnamaldehyde, anethole, anisaldehyde, anisyl alcohol, anisole, methyl anthranilate, acetophenone, benzylacetone, benzaldehyde, ethyl benzoate, benzophenone, benzyl alcohol, benzyl acetate, benzyl benzoate, benzyl formate, benzyl valerate, borneol, bornyl acetate, α-bromostyrene, n-decylaldehyde, n-dodecylaldehyde, eugenol, eugenol methyl ether, eucalyptol, farnesol, fenchone, fenchyl acetate, geranyl acetate, geranyl formate, heliotropin, methyl heptynecarboxylate, heptaldehyde, hydroquinone dimethyl ether, hydroxycinnamaldehyde, hydroxycinnamyl alcohol, indole, iron, isoeugenol, isoeugenol methyl ether, isosafrol, jasmone, camphor, carvacrol, carvone, p-cresol methyl ether, coumarin, p-methoxyacetophenone, methyl n-amyl ketone, methyl methylanthranilate, p-methylacetophenone, methylchavicol, p-methylquinoline, methyl β-naphthyl ketone, methyl-n-nonylacetaldehyde, methyl n-nonyl ketone, muscone, β-naphthol ethyl ether, β-naphthol methyl ether, nerol, nitrobenzene, n-nonylaldehyde, nonyl alcohol, n-octylaldehyde, p-oxyacetophenone, pentadecanolide, β-phenylethyl alcohol, phenylacetaldehyde dimethyl acetal, phenylacetic acid, pulegone, safrol, isoamyl salicylate, methyl salicylate, hexyl salicylate, cyclohexyl salicylate, santalol, skatole, terpineol, thymene, thymol, γ-undelactone, vanillin, veratrumaldehyde, cinnamaldehyde, cinnamyl alcohol, cinnamic acid, ethyl cinnamate and benzyl cinnamate.

The more volatile odorants include, in particular, the lower-boiling odorants of natural or synthetic origin, which may be used alone or in mixtures. Examples of more volatile odorants are alkyl isothiocyanates (alkyl mustard oils), butanedione, limonene, linalool, linalyl acetate and linalyl propionate, menthol, menthone, methyl-n-heptenone, phellandrene, phenylacetaldehyde, terpinyl acetate, citral and citronellal.

The compositions of the invention are suitable for lightening skin and/or hair and can be employed accordingly. The treatment of the skin in this context may be locally limited (e.g., to freckles, age spots or instances of aberrant pigmentation) or extensive (for skin lightening). Keratinic fibers as well, more particularly human hair, can be lightened locally (“streaks effect”) or entirely with the compositions of the invention.

Further provided by the present invention, therefore, is the use of cosmetic or dermatological preparations of the invention against unwanted pigmentation of the skin and/or to treat pigmentation defects; the use of cosmetic or dermatological preparations of the invention to lighten age spots on the skin; and the use of cosmetic or dermatological preparations of the invention to lighten the skin color of human beings, more particularly those of Indian or Asian origin.

The present invention further provides for the use of cosmetic or dermatological preparations of the invention against unwanted pigmentation of the hair and/or to lighten the hair.

The present invention further provides for the use of cosmetic and dermatological preparations of the invention to treat postinflammatory hyperpigmentation.

With regard to preferred embodiments of the uses according to the invention, the same applies, mutatis mutandis, as for the compositions of the invention.

The examples which follow are intended to elucidate the subject matter of the invention in more detail without imposing any restriction on it.

### EXAMPLE SERIES 1

### 1. Oil-in-Water Emulsions

| | 1 | 2 | 3 |
|---|---|---|---|
| Thistle oil | 3.00 | 3.00 | 3.00 |
| Myritol 318 | 5.00 | 5.00 | 5.00 |
| Novata AB | 2.00 | 2.00 | 2.00 |
| Lanette 22 | 1.00 | 1.00 | 1.00 |
| Cutina MD | 2.00 | 2.00 | 2.00 |
| Stenol 1618 | 1.00 | 1.00 | 1.00 |
| Isopropyl stearate | 4.00 | 4.00 | 4.00 |
| Cetiol SB 45 | 2.00 | 2.00 | 2.00 |
| Baysilon M350 | 1.00 | 1.00 | 1.00 |
| Controx KS | 0.05 | 0.05 | 0.05 |
| Propylparaben | 0.20 | 0.20 | 0.20 |
| Dow Corning Fluid 1501 | 1.00 | 1.00 | 1.00 |
| Dry Flo Plus | 1.00 | 1.00 | 1.00 |
| TiO2 | 0.50 | 0.50 | 0.50 |
| Hexanediol | 6.00 | 3.00 | |
| Propylene glycol | 5.00 | 5.00 | 5.00 |
| Glycerine | 5.00 | 3.00 | 3.00 |
| Methylparaben | 0.20 | 0.20 | 0.20 |
| Tego Carbomer | 0.40 | 0.40 | 0.40 |
| DS-HCN | 5.00 | 5.00 | 5.00 |
| Spirulina Extract 3002 | 1.00 | 0.50 | 0.10 |
| Taurine | 1.00 | | |
| Arlatone Dioic Acid | 1.00 | 0.50 | 0.20 |
| Vitamin C | 0.20 | | 1.00 |
| Herbalia Rose HIP | | 0.10 | |
| Parfum | 0.10 | 0.10 | 0.10 |
| Aqua | ad. 100 | ad. 100 | ad. 100 |

### EXAMPLE SERIES 2

| | 1 | 2 | 3 |
|---|---|---|---|
| Cetiol SN | 4.00 | 4.00 | 4.00 |
| Mineral oil | 6.00 | 6.00 | 6.00 |
| Cutina CBS | 2.00 | 2.00 | 2.00 |
| Edenor L2 SM | 1.50 | 1.50 | 1.50 |
| Emulgin B3 | 1.00 | 1.00 | 1.00 |
| Baysilon M 350 | 1.00 | 1.00 | 1.00 |
| Tocopheryl acetate | 0.50 | 0.50 | 0.50 |
| Propylparaben | 0.30 | 0.30 | 0.30 |
| Almond oil | 2.00 | 2.00 | 2.00 |
| Permulen Tr-1 | 0.27 | 0.27 | 0.27 |
| Glycerin | 5.00 | 3.00 | 3.00 |
| Lactic acid 80% | 0.26 | 0.26 | 0.26 |
| Propylene glycol | 5.00 | 5.00 | 5.00 |
| Methylparaben | 0.30 | 0.30 | 0.30 |
| Phenoxyethanol | 0.90 | 0.90 | 0.90 |
| Panthenol | 0.50 | | |
| Laminaria Digitata Extract | 1.00 | | |
| Sodium chloride | 0.05 | 0.05 | 0.05 |
| Seppigel 305 | 0.50 | 0.50 | 0.50 |
| Silk Protein | 0.25 | 0.25 | 0.25 |
| Keltrol SF | 0.20 | 0.20 | 0.20 |
| Arlatone Dioic Acid | 1.00 | 0.50 | 0.20 |
| Solu C10 | | | 1.00 |
| Ascorbyl glucoside | | 0.50 | |
| Ascorbyl phosphate | 0.50 | | |
| Taurine | 1.00 | | |
| Parfum | 0.30 | 0.30 | 0.30 |
| Aqua | ad. 100 | ad. 100 | ad. 100 |

### EXAMPLE SERIES 3

| | 1 | 2 | 3 |
|---|---|---|---|
| Lipoid S75-3 | 0.50 | 0.50 | 0.50 |
| Isopropyl stearate | 4.00 | 4.00 | 4.00 |
| Cetiol B | 2.00 | 2.00 | 2.00 |
| Tocopheryl acetate | 0.50 | 0.50 | 0.50 |
| Cutina MD | 1.00 | 1.00 | 1.00 |
| Lanette 22 | 2.00 | 2.00 | 2.00 |
| Baysilone M 350 | 0.50 | 0.50 | 0.50 |
| Propylparaben | 0.20 | 0.20 | 0.20 |
| Dow Corning 9040 | 1.00 | 1.00 | 1.00 |
| Glycerin | 4.50 | 3.00 | 3.00 |
| Hexanediol | 6.00 | 3.00 | |
| Methylparaben | 0.20 | 0.20 | 0.20 |
| Tego Carbomer 140 | 0.30 | 0.30 | 0.30 |
| DS-HCN | 5.00 | 5.00 | 5.00 |
| Algae extract | 1.00 | | |
| Phytosomes | 0.20 | 0.20 | 0.20 |
| Lipochroman-6 | 0.01 | 0.01 | 0.01 |
| Propylene glycol | 5.00 | 5.00 | 5.00 |
| Matrixyl | 3.00 | | |
| Matrixyl 3000 | | 3.00 | |
| Simugel NS | 1.50 | 1.50 | 1.50 |
| TiO2 | 0.50 | 0.50 | |
| Arlatone Dioic Acid | 1.00 | 0.50 | 0.20 |
| Vitamin C | 1.00 | | 0.50 |
| Ascorbyl tetraisopalmitate | | 0.50 | 0.50 |
| Taurine | | 0.50 | |
| Parfum | 0.35 | 0.35 | 0.35 |
| Aqua | ad. 100 | ad. 100 | ad. 100 |

### EXAMPLE SERIES 4

| | 1 | 2 | 3 | 4 | 5 | 6 | 7 | 8 | 9 | 10 |
|---|---|---|---|---|---|---|---|---|---|---|
| Montanov 68 | 5.00 | 5.00 | 5.00 | 5.00 | 5.00 | 5.00 | 5.00 | 5.00 | 5.00 | 5.00 |
| Myritol 316 | 5.00 | 5.00 | 5.00 | 5.00 | 5.00 | 5.00 | 5.00 | 5.00 | 5.00 | 5.00 |
| Cetiol SB 45 | 0.50 | 0.50 | 0.50 | 0.50 | 0.50 | 0.50 | 0.50 | 0.50 | 0.50 | 0.50 |
| Novata AB | 2.00 | 2.00 | 2.00 | 2.00 | 2.00 | 2.00 | 2.00 | 2.00 | 2.00 | 2.00 |
| Stenol 1618 | 1.00 | 1.00 | 1.00 | 1.00 | 1.00 | 1.00 | 1.00 | 1.00 | 1.00 | 1.00 |
| Baysilon M 350 | 0.50 | 0.50 | 0.50 | 0.50 | 0.50 | 0.50 | 0.50 | 0.50 | 0.50 | 0.50 |
| Tocopheryl acetate | 0.50 | 0.50 | 0.50 | 0.50 | 0.50 | 0.50 | 0.50 | 0.50 | 0.50 | 0.50 |
| Controx KS | 0.25 | 0.25 | 0.25 | 0.25 | 0.25 | 0.25 | 0.25 | 0.25 | 0.25 | 0.25 |
| Parsol SLX | 4.00 | | | | | | | | | |
| Parsol HS | 2.00 | 2.00 | 2.00 | | | | | | | 2.00 |
| Parsol 340 | | | | 5.00 | 5.00 | 5.00 | 5.00 | 5.00 | 5.00 | |
| Uvinul MBC 95 | | | | | | | | 4.00 | 4.00 | |
| Parsol 1789 | 1.80 | 1.80 | 1.80 | | | 2.00 | 2.00 | 2.00 | 2.00 | 2.00 |
| Uvinul T 150 | | 2.50 | | 2.50 | 2.50 | | 3.00 | 2.50 | | 2.50 |
| Uvinul A Plus | | | | | 5.00 | | | | | |
| Uvinul A plus B | | | | 5.00 | | | | | | |
| Titanium dioxide/TiO2 | | | 2.00 | | | 2.00 | | 2.00 | 2.00 | |
| dispersions | | | | | | | | | | |
| Eusolex T-AVO | | | | | | | | | | |
| Tioveil TG | | | | | | | | | | |
| Solaveil CT 100 | | | | | | | | | | |
| Propylparaben | 0.20 | 0.20 | 0.20 | 0.20 | 0.20 | 0.20 | 0.20 | 0.20 | 0.20 | 0.20 |
| Tego Carbomer 140 | 0.50 | 0.50 | 0.50 | 0.50 | 0.50 | 0.50 | 0.50 | 0.50 | 0.50 | 0.50 |
| Hexanediol | 6.00 | 6.00 | 3.00 | 6.00 | 6.00 | 6.00 | 6.00 | 6.00 | 6.00 | 6.00 |
| Talc Pharam G | 0.50 | 0.50 | 0.50 | 0.50 | 0.50 | 0.50 | 0.50 | 0.50 | 0.50 | 0.50 |
| Methylparaben | 0.20 | 0.20 | 0.20 | 0.20 | 0.20 | 0.20 | 0.20 | 0.20 | 0.20 | 0.20 |
| Glycerin | 4.50 | 4.50 | 3.00 | 4.50 | 3.00 | 4.50 | 4.50 | 4.50 | 4.50 | 4.50 |
| Dry Flo Plus | 1.00 | 1.00 | 1.00 | 1.00 | 1.00 | 1.00 | 1.00 | 1.00 | 1.00 | 1.00 |
| Natipide 2 PG | 1.00 | 1.00 | 1.00 | 1.00 | 1.00 | 1.00 | 1.00 | 1.00 | 1.00 | 1.00 |
| Gatuline R/C | 2.00 | 2.00 | 2.00 | 2.00 | 2.00 | 2.00 | 2.00 | 2.00 | 2.00 | 2.00 |
| Hydrolyzed protein | 5.00 | 5.00 | 5.00 | 5.00 | 5.00 | 5.00 | 5.00 | 5.00 | 5.00 | 5.00 |
| DS-HCN | 2.00 | 2.00 | 2.00 | 2.00 | 2.00 | 2.00 | 2.00 | 2.00 | 2.00 | 2.00 |
| Trilon A | 0.10 | 0.10 | 0.10 | 0.10 | 0.10 | 0.10 | 0.10 | 0.10 | 0.10 | 0.10 |
| Phenoxyethanol | 0.40 | 0.40 | 0.40 | 0.40 | 0.40 | 0.40 | 0.40 | 0.40 | 0.40 | 0.40 |
| Taurine | 1.00 | | | 1.00 | 1.00 | 1.00 | 1.00 | 1.00 | 1.00 | 1.00 |
| Arlatone Dioic Acid | 1.00 | 0.50 | 0.10 | 0.20 | 1.00 | 1.00 | 1.00 | 1.00 | 1.00 | 1.00 |
| Ascorbyl phosphate | 0.50 | 1.00 | 1.50 | 1.00 | 0.20 | 1.00 | 1.00 | 1.00 | 1.00 | 1.00 |
| Parfum | 0.40 | 0.40 | 0.40 | 0.40 | 0.40 | 0.40 | 0.40 | 0.40 | 0.40 | 0.40 |
| Aqua | ad. | ad. | ad. | ad. | ad. | ad. | ad. | ad. | ad. | ad. |
| | 100 | 100 | 100 | 100 | 100 | 100 | 100 | 100 | 100 | 100 |

### EXAMPLE SERIES 5

| | 1 | 2 | 3 | 4 | 5 | 6 | 7 | 8 | 9 | 10 | 11 | 12 | 13 |
|---|---|---|---|---|---|---|---|---|---|---|---|---|---|
| Montanav 68 | 5.00 | 5.00 | 5.00 | 5.00 | 5.00 | 5.00 | 5.00 | 5.00 | 5.00 | 5.00 | 5.00 | 5.00 | 5.00 |
| Cetiol SN | 5.00 | 5.00 | 5.00 | 5.00 | 5.00 | 5.00 | 5.00 | 5.00 | 5.00 | 5.00 | 5.00 | 5.00 | 5.00 |
| Caprylic/capric | 5.00 | 5.00 | 5.00 | 5.00 | 5.00 | 5.00 | 5.00 | 5.00 | 5.00 | 5.00 | 5.00 | 5.00 | 5.00 |
| triglyceride | | | | | | | | | | | | | |
| Baysilon M 350 | 1.50 | 1.50 | 1.50 | 1.50 | 1.50 | 1.50 | 1.50 | 1.50 | 1.50 | 1.50 | 1.50 | 1.50 | 1.50 |
| Stenon 1618 | 0.50 | 0.50 | 0.50 | 0.50 | 0.50 | 0.50 | 0.50 | 0.50 | 0.50 | 0.50 | 0.50 | 0.50 | 0.50 |
| Cutina MD | 0.50 | 0.50 | 0.50 | 0.50 | 0.50 | 0.50 | 0.50 | 0.50 | 0.50 | 0.50 | 0.50 | 0.50 | 0.50 |
| Uninul T150 | 1.00 | 1.00 | 1.00 | | | | | | | | | | |
| Controx KS | 0.05 | 0.05 | 0.05 | 0.05 | 0.05 | 0.05 | 0.05 | 0.05 | 0.05 | 0.05 | 0.05 | 0.05 | 0.05 |
| Polyparaben | 0.20 | 0.20 | 0.20 | 0.20 | 0.20 | 0.20 | 0.20 | 0.20 | 0.20 | 0.20 | 0.20 | 0.20 | 0.20 |
| Tocopheryl acetate | 0.50 | 0.50 | 0.50 | 0.50 | 0.50 | 0.50 | 0.50 | 0.50 | 0.50 | 0.50 | 0.50 | 0.50 | 0.50 |
| Tiovell TG | 2.10 | 2.10 | 2.10 | | | | | | | | | | |
| Parsol 1789 | | | | 1.80 | 1.80 | 1.80 | | | 2.00 | 2.00 | 2.00 | | |
| Parsol 340 | | | | | | | 2.00 | 2.00 | 5.00 | 5.00 | 5.00 | | |
| Parsol SLX | | | | 4.00 | | | | | | | | | |
| Parsol HS | | | | 2.00 | 2.00 | 2.00 | | | | | | | |
| Uvinul MBC 95 | | | | | | | | | | | 4.00 | 4.00 | |
| Uvinul T 150 | | | | | 2.50 | | 2.50 | 2.50 | | 3.00 | | 2.50 | 2.50 |
| Uvinul A Plus | | | | | | | | 5.00 | | | | | |
| Uvinul A plus B | | | | | | | 5.00 | | | | | | |
| Titanium dioxide/TiO2 | | | | | | 2.00 | | | 2.00 | | 2.00 | 2.00 | |
| dispersions | | | | | | | | | | | | | |
| Eusolex T-AVO | | | | | | | | | | | | | |
| Tioveil TG | | | | | | | | | | | | | |
| Solaveil CT 100 | | | | | | | | | | | | | |
| Cetiol SB 45 | 1.50 | 1.50 | 1.50 | 1.50 | 1.50 | 1.50 | 1.50 | 1.50 | 1.50 | 1.50 | 1.50 | 1.50 | 1.50 |
| Glycerin | 4.50 | 4.50 | 4.50 | 4.50 | 4.50 | 4.50 | 4.50 | 4.50 | 4.50 | 4.50 | 4.50 | 4.50 | 4.50 |
| Sorbitol | 1.40 | 1.40 | 1.40 | 1.40 | 1.40 | 1.40 | 1.40 | 1.40 | 1.40 | 1.40 | 1.40 | 1.40 | 1.40 |
| Hexanediol | 6.00 | 6.00 | 6.00 | 6.00 | 6.00 | 6.00 | 6.00 | 6.00 | 6.00 | 6.00 | 6.00 | 6.00 | 6.00 |
| Methylparaben | 0.20 | 0.20 | 0.20 | 0.20 | 0.20 | 0.20 | 0.20 | 0.20 | 0.20 | 0.20 | 0.20 | 0.20 | 0.20 |
| Tego Carbomer 140 | 0.40 | 0.40 | 0.40 | 0.40 | 0.40 | 0.40 | 0.40 | 0.40 | 0.40 | 0.40 | 0.40 | 0.40 | 0.40 |
| Talc | 1.30 | 1.30 | 1.30 | 1.30 | 1.30 | 1.30 | 1.30 | 1.30 | 1.30 | 1.30 | 1.30 | 1.30 | 1.30 |
| Phenoxyethanol | 0.40 | 0.40 | 0.40 | 0.40 | 0.40 | 0.40 | 0.40 | 0.40 | 0.40 | 0.40 | 0.40 | 0.40 | 0.40 |
| Ederline L | 2.00 | 2.00 | 2.00 | 2.00 | 2.00 | 2.00 | 2.00 | 2.00 | 2.00 | 2.00 | 2.00 | 2.00 | 2.00 |
| Fucogel 100 | 2.00 | 2.00 | 2.00 | 2.00 | 2.00 | 2.00 | 2.00 | 2.00 | 2.00 | 2.00 | 2.00 | 2.00 | 2.00 |
| Relipidium | 0.50 | 0.50 | 0.50 | 0.5 | 0.5 | 0.5 | 0.5 | 0.5 | 0.5 | 0.5 | 0.5 | 0.5 | 0.5 |
| Taurine | 1.00 | | | 1.00 | 1.00 | 1.00 | 1.00 | 1.00 | 1.00 | 1.00 | 1.00 | 1.00 | 1.00 |
| Arlatone Dioic Acid | 1.00 | 0.20 | 0.50 | 1.00 | 1.00 | 1.00 | 1.00 | 1.00 | 1.00 | 1.00 | 1.00 | 1.00 | 1.00 |
| Ascorbyl phosphate | 0.50 | 1.00 | 0.70 | 1.00 | 1.00 | 1.00 | 1.00 | 1.00 | 1.00 | 1.00 | 1.00 | 1.00 | 1.00 |
| Parfum | 0.40 | 0.40 | 0.40 | 0.40 | 0.40 | 0.40 | 0.40 | 0.40 | 0.40 | 0.40 | 0.40 | 0.40 | 0.40 |
| Aqua | ad. | ad. | ad. | ad. | ad. | ad. | ad. | ad. | ad. | ad. | ad. | ad. | ad. |
| | 100 | 100 | 100 | 100 | 100 | 100 | 100 | 100 | 100 | 100 | 100 | 100 | 100 |

### 2. Water-in-oil Emulsion

| | 1 | 2 | 3 |
|---|---|---|---|
| Lameform TGI | 3.00 | 3.00 | 3.00 |
| PEG-45/Dodecyl Glycol Copolymer | 0.50 | 0.50 | 0.50 |
| Microcrystalline Wax | 3.00 | 3.00 | 3.00 |
| Bis-Diglyceryl Polyacyladipate-2 | 1.00 | 1.00 | 1.000 |
| Paraffin oil | 8.00 | 8.00 | 8.00 |
| Vaseline | 2.00 | 2.00 | 2.00 |
| Vitamin E acetate | 2.00 | 2.00 | 2.00 |
| Methylparaben | 0.30 | 0.30 | 0.30 |
| Propylparaben | 0.30 | 0.30 | 0.30 |
| Isopropyl isostearate | 8.00 | 8.00 | 8.00 |
| Glycerin | 5.00 | 3.00 | 3.00 |
| Mg sulfate | 0.50 | 0.50 | 0.50 |
| Taurine | 1.00 | 0.50 | 0.10 |
| Lactic acid 80% strength | 0.56 | 0.56 | 0.56 |
| Algae extract | 1.00 | | |
| Panthenol | 0.50 | 1.00 | 0.50 |
| Calendula Officinalis Flower Extract | 0.30 | | |
| Arlatone Dioic Acid | 1.00 | 0.20 | 0.50 |
| Ascorbyl phosphate | 0.50 | 0.50 | |
| Solu C10 | | 0.50 | 1.00 |
| Propylene glycol | 0.50 | | |
| Parfum | 0.20 | 0.20 | 0.20 |
| Aqua | ad. 100 | ad. 100 | ad. 100 |

### 3. Cleansing Preparations

### EXAMPLES SERIES 1

| | 1 | 2 | 3 |
|---|---|---|---|
| Dipropylene glycol | 10.00 | 10.00 | 10.00 |
| Chlorhexidine digluconate | 1.00 | 1.00 | 1.00 |
| Synperonic PE7L 64 | 3.00 | 3.00 | 3.00 |
| D-Panthenol | 0.50 | 0.50 | 0.50 |
| Hydagen CMF | 3.00 | 3.00 | 3.00 |
| PEG-40 hydrogenated castor oil/ | 0.50 | 0.50 | 0.50 |
| trideceth 9/propylene glycol | | | |
| Chlorella Vulgaris extract | 0.50 | 0.50 | 0.50 |
| Taurine | 1.00 | 0.50 | 0.10 |
| Arlatone Dioic Acid | 1.00 | 0.50 | 0.20 |
| Herbalia Rose HIP | 0.50 | | 0.20 |
| Ascorbyl phosphate | | 0.50 | 0.50 |
| Parfums | 0.20 | 0.20 | 0.20 |
| Aqua | ad. 100 | ad. 100 | ad. 100 |

### EXAMPLE SERIES 2

| | 1 | 2 | 3 |
|---|---|---|---|
| Carbopol ETD 2001 | 1.40 | 1.40 | 1.40 |
| Sorbitol | 2.10 | 2.10 | 2.10 |
| Sodium benzoate | 0.40 | 0.40 | 0.40 |
| Plantacare 2000UP | 7.50 | 7.50 | 7.50 |
| Cocoamidopropyl betaine | 3.40 | 3.40 | 3.40 |
| Texapon SB 3 | 5.00 | 5.00 | 5.00 |
| Cetiol HE | 0.50 | 0.50 | 0.50 |
| Lamesoft PO 65 | 5.00 | 5.00 | 5.00 |
| Controx KS | 0.05 | 0.05 | 0.05 |
| Ajidew NL 50 | 1.60 | 1.60 | |
| Pantolactone | 1.00 | 1.00 | |
| Triton B | 0.25 | 0.25 | 0.25 |
| Sodium lactate | 1.80 | | |
| D-Panthenol | 0.50 | 0.50 | 0.50 |
| Taurine | 1.00 | 0.10 | 0.50 |
| Arlatone Dioic Acid | 1.00 | 0.20 | 0.50 |
| Vitamin C | 0.50 | | 0.25 |
| Ascorbyl glucoside | | 0.70 | 0.25 |
| Parfum | 0.40 | 0.40 | 0.40 |
| Aqua | ad. 100 | ad. 100 | ad. 100 |

### EXAMPLE SERIES 3

| | 1 | 2 | 3 |
|---|---|---|---|
| Hostapon SCI-65 | 22.50 | 22.50 | 22.50 |
| Steasilk 5GG HT | 4.00 | 4.00 | 4.00 |
| Cutina FS 45 | 6.00 | 6.00 | 6.00 |
| Cocoamidopropyl betaine 40% | 9.50 | 9.50 | 9.50 |
| Na benzoate | 0.40 | 0.40 | 0.40 |
| Sorbitol 70% | 1.00 | 1.00 | 1.00 |
| Glycerin 86% | 1.00 | 1.00 | 1.00 |
| D-Panthenol 75% | 0.13 | 0.13 | 0.13 |
| Taurine | 1.00 | | |
| Arlatone Dioic Acid | 1.00 | 0.20 | 0.50 |
| Vitamin C | 1.00 | | 0.20 |
| Solu C10 | | 2.00 | 1.00 |
| Parfum | 0.30 | 0.30 | 0.30 |
| Aqua | ad. 100 | ad. 100 | ad. 100 |

### EXAMPLE SERIES 4

| | 1 | 2 | 3 |
|---|---|---|---|
| Paraffinum Liquidum | 20.00 | 20.00 | 20.00 |
| Hostaphat KW 340 D | 2.50 | 2.50 | 2.50 |
| Cetearyl alcohol | 1.40 | 1.40 | 1.40 |
| Eumulgin B 1 | 1.70 | 1.70 | 1.70 |
| Vitamin E acetate | 0.25 | 0.25 | 0.25 |
| Propylparaben | 0.20 | 0.20 | 0.20 |
| Glycerin 86% vegetable | 2.00 | 2.00 | 2.00 |
| Hexane-1,6-diol | 5.00 | 5.00 | 5.00 |
| Sorbitol 70% LS DAB | 1.00 | 1.00 | 1.00 |
| Methylparaben | 0.20 | 0.20 | 0.20 |
| Tego Carbomer 140 | 0.30 | 0.30 | 0.30 |
| Keltrol SF | 0.10 | 0.10 | 0.10 |
| D-Panthenol 75% | 0.70 | 0.70 | 0.70 |
| Phenoxyethanol, pure | 0.40 | 0.40 | 0.40 |
| Calcium D-pantothenate | 0.05 | 0.05 | 0.05 |
| Arlatone Dioic Acid | 1.00 | 0.20 | 0.50 |
| Herbalia Rose HIP | 1.00 | | 0.20 |
| Solu C10 | | 2.00 | 1.00 |
| Taurine | 1.00 | | |
| Parfum | 0.25 | 0.25 | 0.25 |
| Aqua | ad. 100 | ad. 100 | ad. 100 |

### 4. Water-in-silicone Emulsion

| | 1 | 2 | 3 |
|---|---|---|---|
| Belsil DM 100 | 2.50 | 2.50 | 2.50 |
| Dow Corning 245 Fluid | 25.00 | 25.00 | 25.00 |
| Abil EM 90 | 2.00 | 2.00 | 2.00 |
| Sodium chloride | 2.00 | 2.00 | 2.00 |
| Parfum | 0.30 | 0.30 | 0.30 |
| Symdiol 68 | 0.30 | 0.30 | 0.30 |
| Phenoxyethanol | 0.40 | 0.40 | 0.40 |
| Taurine | 1.00 | | |
| Arlatone Dioic Acid | 1.00 | 0.20 | 0.10 |
| Ascorbyl phosphate | 0.50 | | 0.50 |
| Ascorbyl tetraisopalmitate | | 0.50 | 0.50 |
| Herbalia Rose HIP | | 0.10 | |
| Aqua | ad. 100 | ad. 100 | ad. 100 |

### Whitening Actives Used.

| | | Supplier/ |
|---|---|---|
| Trade name | INCI name | Manufacturer |
| Arlatone Dioic Acid | Octadecenedioic Acid | Uniqema |
| Vitamin C | Ascorbic Acid | Roche |
| Herbalia Rose HIP | Rose Canina (and) | Cognis |
| | Maltodextrin (and) Silica | |
| Natrium Ascorbyl | Sodium Ascorbyl Phosphate | BASF |
| Phosphate | | |
| VC-IP | Ascorbyl Tetraisopalmitate | Jan Dekker |
| Ascorbic Acid | Ascorbic Acid Glucoside | Siber Hegner |
| 2-Glucoside | | |
| Phyliance | Phytic Acid | Soliance |
| Melaslow | CITRUS UNSHIU (PEEL | Sederma |
| | EXTRACT), GLYCERIN | |
| Lumiskin | CAPRYLIC/CAPRIC | Sederma |
| | TRIGLYCERIDE (AND) | |
| | DIACETYLBOLDINE | |
| Melanostatine DM | WATER, DEXTRAN (AND) | IMPAG |
| | HEXAPEPTIDE-2, PHENONIP | |
| Polyol Licorice | GLYCYRRHIZA CLABRA | Jan Dekker/ |
| P-T 40 | | Nikko |
| Aqua Licorice | GLYCYRRHIZA GLABRA, | Jan Dekker/ |
| Extract P-T | PPG-6-DECYLTETRA | Nikko |
| | DECETH-30, BUTYLENE | |
| | GLYCOL, WATER | |

### Other Actives Used.

| | | Supplier/ |
|---|---|---|
| Trade name | INCI name | Manufacturer |
| Abil EM 90 | Cetyl dimethicone copolyol | Goldschmidt |
| Ajidew NL 50 | AQUA, SODIUM PCA | Ajinomoto |
| Areaumat Samphira | Crithmum maritimum | Codif |
| Baysilon M350 | Silicone oil | Bayer |
| Belsil DM 100 | Dimethicone | Wacker |
| Carbopol ETD 2001 | Carbomer | Noveon |
| Cetiol ® B | Dibutyl Adipate | Cognis |
| Cetiol ® HE | PEG-7 Glyceryl Cocoate | Cognis |
| Cetiol ® SB 45 | Butyrospermum Parkii (Shea butter) | Cognis |
| Cetiol ® SN | Cetearyl isononanoate | Cognis |
| Controx KS | Tocopherol, Hydrogenated Palm | Cognis |
| | Glycerides Citrate | |
| Cutina CBS | Glyceryl Stearate/Cetearyl Alcohol/ | Cognis |
| | Cetyl Palmitate/Cocoglycerides | |
| Cutina MD | Glyceryl Stearate | Cognis |
| Cutina FS 45 | Stearic Acid & Palmitic Acid | Cognis |
| Dow Corning 9040 | Cyclopentasiloxane (and) | Dow Corning |
| | Dimethicone Crosspolymer (and) | |
| | Cyclohexasiloxane | |
| Dow Corning 245 Fluid | Cyclomethicone | Dow Corning |
| Dow Corning 1501 Fluid | Cyclopentasiloxane (and) | Dow Corning |
| | dimethiconol | |
| Dry Flo Plus | Aluminium Starch Octenylsuccinate | National Starch |
| DS-HCN | Water, Dimethylsilanol | Exsymol |
| | Hyaluronate | |
| Edenor L2 SM | Palmitic/Stearic acid | |
| Ederline L | Hexyldecanol, Pyrus Malus (Apple) | Impag |
| | Fruit Extract | |
| Eumulgin B1 | Ceteareth-10 | Cognis |
| Eumulgin B3 | Ceteareth-30 | Cognis |
| Fucogel 100 | Biosaccharide Gum-1 | Amchem |
| Gatuline R/C | Fagus Silvatica (Beech | Gattefosse |
| | Tree Buds) Extract | |
| Hostaphat KW 340 D | Mono, di and | Clariant |
| | tri(alkyltetraglycolether)-o- | |
| | Phosphoric acid esters | |
| Hostapon SCI-65 | Sodiuim Cocoyl Isethionate | Clariant |
| Hydagen CMF | Chitosan Glycolate | Cognis |
| Keltrol SF | Xanthan Gum | Kelco |
| Lameform TGI | Polyglyceryl-3 Diisostearate | Cognis |
| Lamesoft PO 65 | Coco-Glucoside & Glyceryl Oleate | Cognis |
| Lanette 22 | Behenyl Alcohol | Cognis |
| Lipochroman-6 | Dimethylmethoxy Chromanol | Centerchem |
| Lipoid S75-3 | Hydrogenated Lecithin | Lipoid GmbH |
| Matrixyl | Aqua, Palmitoyl Pentapeptide-3 | Sederma |
| Matrixyl 3000 | Glycerin, Aqua, Butylene Glycol, | Sederma |
| | Carbomer, Polysorbate 20, | |
| | Palmitoyl Oligopeptide, | |
| | Palmitoyl Tetrapeptide-1 | |
| Montanov 68 | Cetearyl Alcohol and Cetearyl | Seppic |
| | Glucoside | |
| Myritol 318 | Caprylic/Capric Triglyceride | Cognis |
| Natipide 2 PG | Aqua (and) Lecithin (and) | Nattermann |
| | Propylene Glycol | |
| Neo Heliopan AP | MENTHYL ANTHRANILATE | Haarmann & Reimer |
| Novata AB | Cocoglycerides | Cognis |
| Parsol SLX | Polysilicone-15 | Hoffman LaRoche |
| Parsol HS | Phenylbenzimidazole Sulfonic | Hoffman LaRoche |
| | Acid | |
| Parsol 340 | octocrylene | Hoffman LaRoche |
| Parsol 1789 | BUTYL METHOXY-DIBENZOYL- | Hoffman LaRoche |
| | METHANE | |
| Pemulen TR 1 | Acrylates/C10-30 Alkyl | Noveon |
| | Acrylate Crosspolymer | |
| Plantacare 2000 UP | Decyl Glucoside | Cognis |
| Relipidium | Hydrolysed yeast protein, | Coletica |
| | Butylene glycol, Methylparaben | |
| Sepigel ® 305 | Polyacrylamide, C₁₃-C₁₄ Isoparaffin, | SEPPIC |
| | Laureth-7 | |
| Simulgel NS | Hydroxyethyl Acrylate/Sodium | Seppic |
| | Acryloyldimethyl Taurate | |
| | Copolymer/Squalane/Polysorbate 60 | |
| Solu C 10 | Ascorbic acid | BASF |
| Spirulina Extrakt 3002 | Water, Algae Extract | Interorgana |
| Steasilk 5GG HT | Talc | Luzenac NV |
| Stenol 1618 | Cetearyl Alcohol | Cognis |
| Symdiol 68 | 1,2-Octanediol, 1,2-Hexanediol | Symrise |
| Synperonic PE/L 64 | Poloxamer 184 | Uniqema |
| Tego Carbomer | Carbomer | Goldschmidt |
| Texapon SB 3 | Disodium Laureth sulfosuccinate | Cognis |
| Tioveil TG | Caprylic/Capric Triglyceride (and) | Uniqema |
| | Titanium Dioxide (and) Alumina (and) | |
| | Polyhydroxystearic Acid (and) Silica | |
| Trilon A | Trisodium EDTA | BASF |
| Triton B | Tetrasodium EDTA | BASF |
| Uvinul T 150 | ethylhexyl triazone | BASF |
| Uvinul MBC 95 | Methylbenzylidene Camphor | BASF |

1. A cosmetic or dermatological preparation comprising from 0.01% to 5% by weight of a dicarboxylic acid selected from the group consisting of 8-hexadecene-1,16-dicarboxylic acid, decanedioic acid, nonanedioic acid and combinations thereof and from 0.01% to 5% by weight of ascorbic acid, salts of ascorbic acid, ascorbic acid derivatives and combinations thereof.

2. The cosmetic or dermatological preparation of claim 1 wherein the amount of 8-hexadecene-1,16-dicarboxylic acid is from 0.05% to 3.0% by weight.

3. The cosmetic or dermatological preparation of claim 1 wherein the amount of ascorbic acid, salts of ascorbic acid, ascorbic acid derivatives and combinations thereof is from 0.05% to 3.0% by weight.

4. The cosmetic or dermatological preparation of claim 1 wherein the weight ratio of 8-hexadecene-1,16-dicarboxylic acid to ascorbic acid and/or salts of ascorbic acid and/or ascorbic acid derivatives is from 5:1 to 1:15.

5. The cosmetic or dermatological preparation of claim 1 comprising ascorbyl phosphate, ascorbyl glucoside, ascobyl tetraisopalmitate or combinations thereof.

6. The cosmetic or dermatological preparation of claim 1 wherein the preparation is free of retinol and retinol derivatives.

7. The cosmetic or dermatological preparation of claim 1 further comprising from 0.5% to 20.0% by weight of one or more compounds selected from the group consisting of triethylhexanone, glyceryl isostearate, propylene glycol isostearate, isopropyl isostearate, ethylhexyl palmitate, ethylhexyl isostearate, squalenes, and triisostearin.

8. The cosmetic or dermatological preparation of claim 7 wherein the compound is propylene glycol isostearate.

9. The cosmetic or dermatological preparation of claim 1 further comprising from 0.5% to 2.5% by weight of 2-aminoethanesulfonic acid.

10. The cosmetic or dermatological preparation of claim 1 further comprising from 0.5% to 2.5% by weight of ethylhexyl triazone.

11. The cosmetic or dermatological preparation of claim 1 further comprising from 0.5% to 2.5% by weight of butylmethoxydibenzoylmethane, diethylaminohydroxybenzoylhexyl benzoate or a combination thereof.

12. The cosmetic or dermatological preparation of claim 1 further comprising from 0.5% to 2.5% by weight of titanium dioxide.

13. The cosmetic or dermatological preparation of claim 1 further comprising from 0.5% to 2.5% by weight of a composition selected from the group consisting of: (a) glycyrrhiza glabra, (b) glycyrrhiza glabra, ppg-6-decyltetradeceth-30, butylene glycol, and water; (c) citrus unshiu and glycerin; (d) phytic acid; (e) caprylic/capric triglyceride and diacetylboldine; (f) water, dextran and hexapeptide-2, phenonip and combinations of (a)-(f).

14. The cosmetic or dermatological preparation of claim 1 further comprising from of 0.1% to 10% by weight of a silicone.

15. The cosmetic or dermatological preparation of claim 1 further comprising a silicone is a compound of the formula I
(CH₃)₃Si—[O—Si(CH₃)₂]ₓ—O—Si(CH₃)₃ (I),
wherein x is a number from 0 to 100.

16. The cosmetic or dermatological preparation of claim 1 further comprising an amino-functional silicone of the formula (II)
R′ₐG₃₋ₐ-Si(OSiG₂)ₙ—(OSiG_{b}R′_{2-b})ₘ—O—SiG₃₋ₐ-R′ₐ (II)
wherein:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a is a number between 0 and 3;
b is a number between 0 and 1;
m and n are numbers whose sum (m+n) is between 1 and 2000;
R′ is a monovalent radical selected from
-Q-N(R″)—CH₂—CH₂—N(R″)₂
-Q-N(R″)₂
-Q-N⁺(R″)₃A⁻
-Q-N⁺H(R″)₂A⁻
-Q-N⁺H₂(R″)A⁻
-Q-N(R″)—CH₂—CH₂—N⁺R″H₂A⁻,
wherein each Q is a chemical bond, —CH₂, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
wherein R″ is independently selected from the group consisting of —H, -phenyl, -benzyl, —CH₂—CH(CH₃)Ph, the C₁₋₂₀-alkyl radicals, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and wherein A is an anion selected from chloride, bromide, iodide or methosulfate.

17. The cosmetic or dermatological preparation of claim 1 further comprising a silicone of the formula III 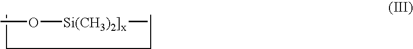
wherein x is a number from 0 to 200.

18. The cosmetic or dermatological preparation of claim 1 further comprising a silicone of the formula IV
R₃Si—[O—Si R₂]ₓ—(CH₂)ₙ-[O—Si R₂]_{y}—O—Si R₃ (IV)
in which R stands for identical or different radicals from the group —H, -phenyl, -benzyl, —CH₂—CH(CH₃)Ph, the C₁₋₂₀-alkyl radicals, preferably
—CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y are each a number from 0 to 200, preferably from 0 to 10, more preferably from 0 to 10, and more particularly 0, 1, 2, 3, 4, 5 or 6, and n is a number from 0 to 10, preferably from 1 to 8, and more particularly is 2, 3, 4, 5 or 6.

19. The cosmetic or dermatological preparation of claim 1 further comprising a water-soluble silicone.
